(19) United States
(12) Patent Application Publication
(10) Pub. No.: US 2002/0082262 A1
(43) Pub. Date: Jun. 27, 2002

(54) Substituted bicyclic compounds for treating multidrug resistance

(76) Inventors: Charles Raymond Degenhardt, Cincinnati, OH (US); David Joseph Eickhoff, Edgewood, KY (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/740,642

(22) Filed: Dec. 19, 2000

Related U.S. Application Data
(60) Provisional application No. 60/241,127, filed on Oct. 17, 2000.

(51) Int. Cl. A61K 31/50; A61K 31/501; A61K 31/497; A61K 31/445; C07D 401/00

(52) U.S. Cl. 514/252.01; 514/253.06; 514/316; 514/253.01; 544/360

(57) ABSTRACT

Substituted bicyclic compounds for treating multidrug resistance are disclosed. Compositions and methods of use for the substituted bicyclic compounds are disclosed. Suitable substituted bicyclic compounds include:

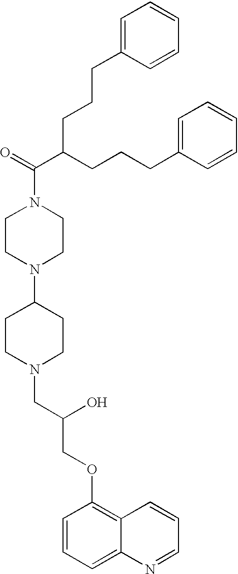

### FIELD OF THE INVENTION

This invention relates to compounds for treating multidrug resistance and methods for their preparation and use. More particularly, this invention relates to substituted bicyclic compounds that regulate the cellular transport proteins P-glycoprotein and MRP1 , which are the proteins believed to be largely responsible for causing multidrug resistance in cancer patients.

### BACKGROUND OF THE INVENTION

“Drug resistance” means a circumstance when a disease (e.g., cancer) does not respond to a therapeutic agent. Drug resistance can be intrinsic, which means that the disease has never been responsive to the therapeutic agent, or acquired, which means that the disease ceases responding to the agent or agents to which the disease had previously been responsive. “Multidrug resistance” is a type of drug resistance wherein a disease is resistant to a variety of drugs that can be functionally unrelated, structurally unrelated, or both. Multidrug resistance is a problem associated with cancer and other conditions, such as bacterial, viral, protozoal, and fungal diseases.

One cause of multidrug resistance in cancer patients is that many cancer cells express high levels of the transmembrane transport proteins, such as Pleiotropic-glycoprotein (also known as Pgp, P-glycoprotein, gp-170, or MDR1) and MRP1 (see Borst, P., “Multidrug resistance: A solvable problem?” Annals of Oncology, 10, suppl. 4, pp. S162-S164 (1999)). In adenosine-triphosphate driven processes, these transport proteins export hydrophobic compounds (such as vinblastine, daunorubicin, doxorubicin, etoposide, vincristine, and TAXOL®, which are cytotoxic drugs useful for treating cancer) from the cell in an effort to protect the cell from harm. The transport proteins remove the compounds from the cell prior to their having a lethal effect on the cell (see Legrand, et. al, “Simultaneous Activity of MRP1 and Pgp Is Correlated With In Vitro Resistance to Daunorubicin and With In Vivo Resistance in Adult Acute Myeloid Leukemia”, Blood, Vol. 94, No. 3, pp. 1046-1056 (1999); and Zhu, B. T.; “A Novel Hypothesis for the Mechanism of Action of P-glycoprotein as a Multidrug Transporter,” Molecular Carcinogenesis 25, pp.1-14 (1999)). Although it is not currently known which of these two classes of proteins is more important for multidrug resistance, and indeed it may be that the class (or classes) of protein which is important depends on the type of cancer and the particular drug or drugs used to treat the cancer, Pgp is known to be highly expressed in approximately 50% of human cancers which require drug therapy. Consequently, Pgp is believed to be a major cause of multidrug resistance.

Other types of multidrug resistance, such as antibacterial, antiviral, and antifungal multidrug resistance may also be caused by the action of transport proteins that are similar to Pgp, and others (see “Annual Reports on Medicinal Chemistry—33; Section III Cancer and Infectious Diseases” ed. Plattner, J., Academic Press, Ch. 12, pp. 121 -130 (1998)).

Furthermore, Pgp is also expressed at high levels in the gastrointestinal tract, liver, kidneys, and brain, and therefore Pgp represents a major pharmacological barrier to the bioavailability of many drugs (see Amudkar, et. al in “Biochemical, Cellular, and Pharmacological Aspects of the Multidrug Transporter,” Annu. Rev. Pharmacol. Toxicol, 39, pp. 361-398 (1999)). For example, the oral bioavailability of many nutrients and drugs is negatively affected by the action of Pgp present in the gastrointestinal tract. “Oral bioavailability” means the ability of a drug or nutrient that is administered orally to be transported across the gastrointestinal tract and enter into the bloodstream. In addition, penetration of many drugs through the blood-brain barrier is adversely affected by Pgp.

### SUMMARY OF THE INVENTION

This invention relates to novel compounds useful in treating or preventing multidrug resistance (“MDR”). More specifically, these compounds are useful in treating or preventing P-glycoprotein-mediated MDR and MRP1 -mediated MDR. This invention further relates to compositions comprising these compounds. This invention further relates to methods for the preparation and use of the compounds and compositions. The compounds and compositions of this invention are well suited for treatment of multidrug resistant cells, for prevention of the development of multidrug resistance, and for use in multidrug resistant chemotherapies.

### DETAILED DESCRIPTION OF THE INVENTION

Publications and patents are referred to throughout this disclosure. All U.S. Patents cited herein are hereby incorporated by reference.

All percentages, ratios, and proportions used herein are by weight unless otherwise specified.

### Definitions and Usage of Terms

The following is a list of definitions, as used herein.

“Aromatic group” means a group having a monocyclic or polycyclic ring structure. Monocyclic aromatic groups contain 4 to 10 carbon atoms, preferably 4 to 7 carbon atoms, and more preferably 4 to 6 carbon atoms in the ring. Preferred polycyclic ring structures have two or three rings. Polycyclic structures having two rings typically have 8 to 12 carbon atoms, preferably 8 to 10 carbon atoms in the rings. Polycyclic aromatic groups include groups wherein at least one, but not all, of the rings are aromatic.

“Carbocyclic group” means a saturated or unsaturated hydrocarbon ring. Carbocyclic groups are not aromatic. Carbocyclic groups are monocyclic or polycyclic. Polycyclic carbocyclic groups can be fused, spiro, or bridged ring systems. Monocyclic carbocyclic groups contain 4 to 10 carbon atoms, preferably 4 to 7 carbon atoms, and more preferably 5 to 6 carbon atoms in the ring. Bicyclic carbocyclic groups contain 8 to 12 carbon atoms, preferably 9 to 10 carbon atoms in the rings.

“Carrier” means one or more substances that are suitable for administration to a subject (i.e., mammal) and that can be combined with the active compound according to this invention. Carrier includes solid and liquid diluents, hydrotropes, surface-active agents, and encapsulating substances.

“Chemosensitizing agent” means a noncytotoxic compound that sensitizes drug resistant cells to the action of cytotoxic drugs. As used in this application, the term “chemosensitizing agent”, excludes the active compounds of this invention.

“Halogen atom” means F, Cl, Br, or I.

“Heteroaromatic group” means an aromatic group containing carbon and 1 to 4 heteroatoms in the ring. Monocyclic heteroaromatic groups contain 4 to 10 member atoms, preferably 4 to 7 member atoms, and more preferably 4 to 6 member atoms in the ring. Preferred polycyclic ring structures have two or three rings. Polycyclic structures having two rings typically have 8 to 12 member atoms, preferably 8 to 10 member atoms in the rings. Polycyclic heteroaromatic groups include groups wherein at least one, but not all, of the rings are heteroaromatic.

“Heteroatom” means an atom other than carbon e.g., in the ring of a heterocyclic group or the chain of a heterogeneous group. Preferably, heteroatoms are selected from the group consisting of sulfur, phosphorous, nitrogen and oxygen atoms. Groups containing more than one heteroatom may contain different heteroatoms.

“Heterocyclic group” means a saturated or unsaturated ring structure containing carbon atoms and 1 or more heteroatoms in the ring. Heterocyclic groups are not aromatic. Heterocyclic groups are monocyclic or polycyclic. Polycyclic heteroaromatic groups can be fused, spiro, or bridged ring systems. Monocyclic heterocyclic groups contain 4 to 10 member atoms (i.e., including both carbon atoms and at least 1 heteroatom), preferably 4 to 7, and more preferably 5 to 6 in the ring. Bicyclic heterocyclic groups contain 8 to 18 member atoms, preferably 9 or 10 in the rings.

“Heterogeneous group” means a saturated or unsaturated chain of non-hydrogen member atoms comprising carbon atoms and at least one heteroatom. Heterogeneous groups typically have 1 to 25 member atoms. Preferably, the chain contains 1 to 12 member atoms, more preferably 1 to 10 , and most preferably 1 to 6. The chain may be linear or branched. Preferred branched heterogeneous groups have one or two branches, preferably one branch. Preferred heterogeneous groups are saturated. Unsaturated heterogeneous groups have one or more double bonds, one or more triple bonds, or both. Preferred unsaturated heterogeneous groups have one or two double bonds or one triple bond. More preferably, the unsaturated heterogeneous group has one double bond.

“Hydrocarbon group” means a chain of 1 to 25 carbon atoms, preferably 1 to 12 carbon atoms, more preferably 1 to 10 carbon atoms, and most preferably 1 to 8 carbon atoms. Hydrocarbon groups may have a linear or branched chain structure. Preferred hydrocarbon groups have one or two branches, preferably 1 branch. Preferred hydrocarbon groups are saturated. Unsaturated hydrocarbon groups have one or more double bonds, one or more triple bonds, or combinations thereof. Preferred unsaturated hydrocarbon groups have one or two double bonds or one triple bond; more preferred unsaturated hydrocarbon groups have one double bond.

“IC₅₀” means concentration of drug required to produce a 50% inhibition of growth of cancer cells or 50% inhibition of activity.

“MDR” means multidrug resistance.

“Parenteral” as used herein includes subcutaneous, intravenous, intramuscular, intraarticular, intrasynovial, intrasternal, intrathecal, intrahepatic, intralesional and intracranial injection or infusion techniques.

“Pgp” means P-glycoprotein.

“Pharmaceutically acceptable” means suitable for use in a human or other mammal.

“Protecting group” is a group that replaces the active hydrogen of a —OH, —COOH, or —NH₂ moiety thus preventing undesired side reaction at the moiety. Use of protecting groups in organic synthesis is well known in the art. Examples of protecting groups are found in Protecting Groups in Organic Synthesis by Greene, T. W. and Wuts, P. G. M., 2nd ed., Wiley & Sons, Inc., 1991. Preferred protecting groups for hydroxyl moieties include silyl ethers, alkoxymethyl ethers, tetrahydropyranyl, tetrahydrofuranyl, esters, and substituted or unsubstituted benzyl ethers. Other preferred protecting groups include carbamates.

“Subject” means a living vertebrate animal such as a mammal (preferably human).

“Substituted aromatic group” means an aromatic group wherein 1 or more of the hydrogen atoms bonded to carbon atoms in the ring have been replaced with other substituents. Preferred substituents include hydrocarbon groups such as methyl groups and heterogeneous groups including alkoxy groups such as methoxy groups. The substituents may be substituted at the ortho, meta, or para position on the ring, or any combination thereof.

“Substituted carbocyclic group” means a carbocyclic group wherein 1 or more hydrogen atoms bonded to carbon atoms in the ring have been replaced with other substituents. Preferred substituents include hydrocarbon groups such as alkyl groups (e.g, methyl groups) and heterogeneous groups such as alkoxy groups (e.g., methoxy groups).

“Substituted heteroaromatic group” means a heteroaromatic group wherein 1 or more hydrogen atoms bonded to carbon atoms in the ring have been replaced with other substituents. Preferred substituents include monovalent hydrocarbon groups including alkyl groups such as methyl groups and monovalent heterogeneous groups including alkoxy groups such as methoxy groups.

“Substituted heterocyclic group” means a heterocyclic group wherein 1 or more hydrogen atoms bonded to carbon atoms in the ring have been replaced with other substituents. Preferred substituents include monovalent hydrocarbon groups including alkyl groups such as methyl groups and monovalent heterogeneous groups including alkoxy groups such as methoxy groups. Substituted heterocyclic groups are not aromatic.

“Substituted heterogeneous group” means a heterogeneous group, wherein 1 or more of the hydrogen atoms bonded to carbon atoms in the chain have been replaced with other substituents. Preferred substituents include monovalent hydrocarbon groups including alkyl groups such as methyl groups and monovalent heterogeneous groups including alkoxy groups such as methoxy groups.

“Substituted hydrocarbon group” means a hydrocarbon group wherein 1 or more of the hydrogen atoms bonded to carbon atoms in the chain have been replaced with other substituents. Preferred substituents include monovalent aromatic groups, monovalent substituted aromatic groups, monovalent hydrocarbon groups including alkyl groups such as methyl groups, monovalent substituted hydrocarbon groups such as benzyl, and monovalent heterogeneous groups including alkoxy groups such as methoxy groups.

“Substrate potential” means the likelihood that a compound for use in treating multidrug resistance will be transported out of a cell by cellular transport proteins before effectively preventing or reversing multidrug resistance.

“Transport protein” means a protein that acts to remove cytotoxic substances from cells through the cell membrane. Transport protein includes P-glycoprotein, MRP1 , and others.

“Treating multidrug resistance” means preventing multidrug resistance from developing in nonresistant cells, increasing or restoring sensitivity of multidrug resistant cells to therapeutic or prophylactic agents, or both.

“Treating” means 1) preventing a disease (i.e., causing the clinical symptoms of the disease not to develop), 2) inhibiting the disease (i.e., arresting the development of clinical symptoms of the disease), 3) relieving the disease (i.e., causing regression of the clinical symptoms), and combinations thereof.

“Wax” means a lower-melting organic mixture or compound of high molecular weight, solid at room temperature and generally similar in formulation to fats and oils except that they contain no glycerides.

### Active Compounds Used in this Invention

The active compounds of this invention are substituted bicyclic heterocyclic compounds. The active compounds can have the structure:

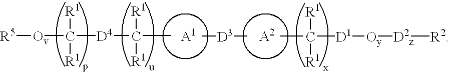

Groups A¹ and A² are each independently selected from the group consisting of carbocyclic groups, substituted carbocyclic groups, heterocyclic groups, substituted heterocyclic groups, aromatic groups, substituted aromatic groups, heteroaromatic groups, and substituted heteroaromatic groups. Groups A¹ and A² typically each have 4 to 9 member atoms, preferably 4 to 7 member atoms, more preferably 5 to 6 member atoms. Groups A¹ and A² are preferably each monocyclic. Groups A¹ and A² are preferably each independently selected from the group consisting of heterocyclic groups and substituted heterocyclic groups. Preferred heterocyclic groups and substituted heterocyclic groups have about 1 to about 2 heteroatoms. Preferred heterocyclic groups include piperazine and piperidine.

Each R¹ is independently selected from the group consisting of a hydrogen atom, a hydroxyl group, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group. R¹ is preferably a hydrogen atom or a hydroxyl group.

The subscript x is 0 to about 10 , preferably 0 to about 1.

R² is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group. R² is preferably selected from the group consisting of a hydrocarbon group, a substituted hydrocarbon group, a heterogeneous group, a substituted heterogeneous group, an aromatic group, a substituted aromatic group, a heteroaromatic group, and a substituted heteroaromatic group. More preferably, R² is a substituted hydrocarbon group or a substituted heterogeneous group, wherein said group is substituted with a group selected from the group consisting of an aromatic group, a substituted aromatic group, a heteroaromatic group, and a substituted heteroaromatic group.

In a preferred embodiment of the invention, R² is selected from the group consisting of:

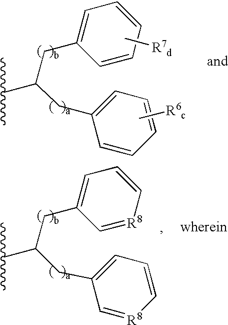

wherein [symbol] denotes a point of attachment, a is at least about 2, b is at least about 2, c is about 1 to about 3, and d is about 1 to about 3. Preferably, a and b are each about 3 to about 10. More preferably, a and b are each about 3.

R⁶ and R¹ are each independently selected from the group consisting of hydrocarbon groups and substituted hydrocarbon groups. Preferably, R⁶ and R⁷ are substituted hydrocarbon groups such as alkoxy groups. Preferred alkoxy groups include methoxy, ethoxy, propoxy, and butoxy.

Each R¹ is independently selected from the group consisting of CH and a heteroatom. Preferably, the heteroatom is nitrogen. More preferably, each R⁸ is CH.

Groups D¹ and D² are each independently selected from the group consisting of—C(O)— and —NR³—,

wherein R³ is selected from the group consisting of a hydrogen atom and R², and with the proviso that optionally, R² and R³ may be bonded together to form a ring structure selected from the group consisting of heterocyclic groups and substituted heterocyclic groups; and

y is 0 or 1 and z is 0 or 1. Preferably, y is 0 and z is 1.

In one embodiment of the invention, R² and R³ are bonded together and the ring structure has 5 to 6 members. Preferably, the ring structure formed by R² and R³ is a substituted heterocyclic group, wherein the substituted heterocyclic group is substituted with a group selected from the group consisting of an aromatic group; a substituted aromatic group; a heteroaromatic group; a substituted heteroaromatic group; a substituted hydrocarbon group, wherein the substituted hydrocarbon group is substituted with a group selected from the group consisting of an aromatic group, a substituted aromatic group, a heteroaromatic group, and a substituted heteroaromatic group; and a substituted heterogeneous group, wherein the substituted heterogeneous group is substituted with a group selected from the group consisting of an aromatic group, a substituted aromatic group, a heteroaromatic group, and a substituted heteroaromatic group.

In an alternative embodiment of the invention, D¹ is —C(O)— and D² is —NR³—. In this embodiment, preferably R³ is selected from the group consisting of a hydrogen atom and a hydrocarbon group.

In an alternative embodiment of the invention, D¹ is —C(O)—, y is 1, and z is 0.

In an alternative embodiment of the invention, D¹ is —NR³— and D² is —C(O)—. In this embodiment, preferably R³ is selected from the group consisting of a hydrogen atom and a hydrocarbon group.

In a preferred embodiment of the invention, D¹ is —C(O)—, y is 0, and z is 0. In this embodiment, x is preferably 0.

D³ is selected from the group consisting of a chemical bond, hydrocarbon groups, substituted hydrocarbon groups, heterogeneous groups, and substituted heterogeneous groups; with the proviso that substituted hydrocarbon groups for D³ are not hydrocarbon groups having only one —C(O)— group. (For example, D³ is not —C(O)— or —CH₂—C(O)—.) D³ is preferably selected from the group consisting of a bond, —(CR¹₂)_{w}—NR⁴—(CR¹₂)_{w}—, —(CR¹₂)₂—C(O)—(CR¹₂)_{w}—NR⁴—(CR¹₂)_{w}—, and —(CR¹ ₂)₂—C(O)—(CR¹₂)_{w}—NR⁴—(CR¹₂)_{w}—C(O)—(CR¹₂)_{w}; wherein each w is independently 0 to about 10, and R⁴ is selected from the group consisting of a hydrogen atom, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group. Preferably, R⁴ is a hydrogen atom. Preferably, each w is 0. More preferably, D³ is a bond.

In the formula above, u is 0 to about 10, p is 0 to about 10, and v is 0 or 1. Preferably, u is about 1 to about 3. Preferably, p is about 1 to about 3. Preferably, v is 1 More preferably, u is about 1 and p is about 1.

D⁴ is selected from the group consisting of —S(O)₂—, —C(O)—, and -CR¹(OH)—. D⁴ is preferably —CR¹(OH)—.

R⁵ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group.

Preferably, R⁵ is selected from the group consisting of an aromatic group; a substituted aromatic group; a heteroaromatic group; a substituted heteroaromatic group; a hydrocarbon group; a substituted hydrocarbon group, wherein the substituted hydrocarbon group is substituted with a group selected from the group consisting of an aromatic group, a substituted aromatic group, a heteroaromatic group, and a substituted heteroaromatic group; and a substituted heterogenous group, wherein the substituted heterogenous group is substituted with a group selected from the group consisting of an aromatic group, a substituted aromatic group, a heteroaromatic group, and a substituted heteroaromatic group.

More preferably, R⁵ is a heteroaromatic group of the formula:

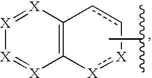

wherein each X is independently selected from the group consisting of CH and a heteroatom, with the proviso that at least one X is a heteroatom. The heteroatom is preferably nitrogen. Preferably, one X is a heteroatom. Examples of heteroaromatic groups for R⁵ include quinolyl and isoquinolyl groups. Preferred quinolyl groups for R⁵ include 4-quinolyl, 5-quinolyl, 6-quinolyl, 7-quinolyl, and 8-quinolyl. More preferably, R⁵ is 5-quinolyl.

In one embodiment of the invention, preferably p is 0, and D⁴ is —SO₂—.

In a preferred embodiment of the invention, D⁴ is —CR¹(OH)— and v is 1.

Examples of compounds having the structure above are shown below in Table 1.

TABLE 1
Example Compounds

| | |
|---|---|
| 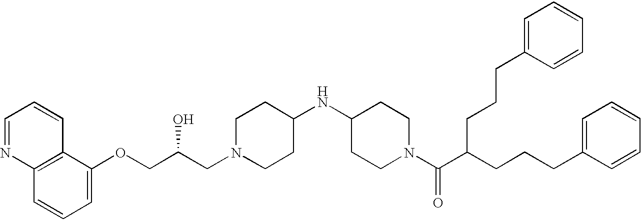 | 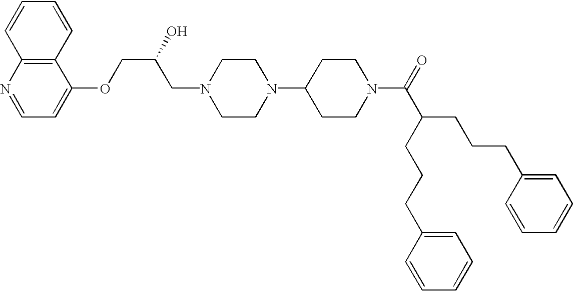 |
| 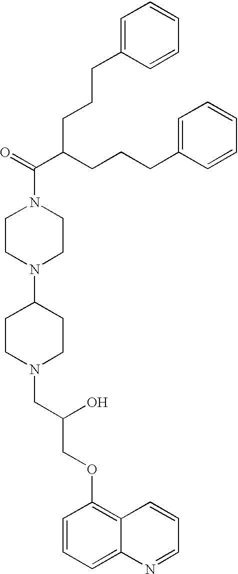 | 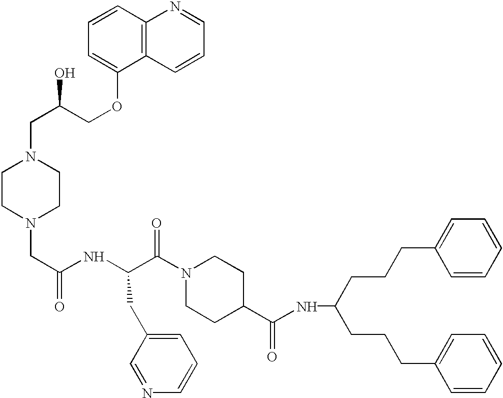 |
| 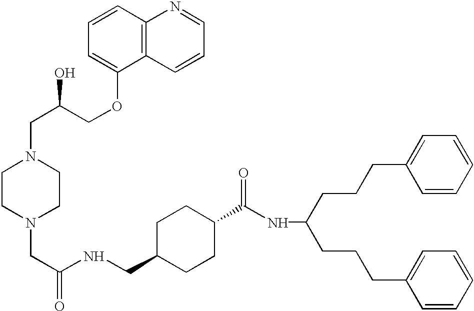 | |

Alternatively, the compound may be an optical isomer, a diastereomer, an enantiomer, a pharmaceutically-acceptable salt, a biohydrolyzable amide, a biohydrolyzable ester, and a biohydrolyzable imide of the structure, or combinations thereof.

The active compound of this invention inhibits at least one transport protein. The active compound preferably inhibits Pgp or MRP1. More preferably, the active compound inhibits both Pgp and MRP1. In a preferred embodiment of this invention, the active compound inhibits Pgp and has low substrate potential for Pgp. In an alternative preferred embodiment, the active compound inhibits MRP1 and has low substrate potential for MRP1. In the most preferred embodiment of this invention, the active compound inhibits both Pgp and MRP1 and the active compound has low substrate potential for both Pgp and MRP1.

The degree to which a compound inhibits a transport protein can be measured by quantitating the effectiveness of the compound toward restoring drug sensitivity to multidrug resistant cells. Methods for quantitating the effectiveness of the active compounds toward restoring drug sensitivity are readily available to one skilled in the art without undue experimentation (see U.S. Pat. Nos. 5,935,954 and 5,272,159, which are hereby incorporated by reference for the purpose of disclosing these methods). Any assay known to measure the restoration of the anti-proliferative activity of a drug may be employed to test the compounds of this invention. These assays use cell lines resistant to particular drugs, and characterized by the presence of one or both of Pgp and MRP1. These cell lines include L1210, HL60, P388, CHO, and MCF7. Alternatively, resistant cell lines can be developed by methods readily available to one of ordinary skill in the art without undue experimentation (see Chaudhary, et al., “Induction of Multidrug Resistance in Human Cells by Transient Exposure to Different Chemotherapeutic Agents,” Journal of the National Cancer Institute, Vol. 85, No. 8, pp. 632-639 (1993)). The cell line is then exposed to compounds of this invention in the presence or absence of the drug to which it is resistant, such as TAXOL®. The viability of the cells treated with both the active compound and the drug can then be compared to the viability of the cells treated only with the drug.

The active compound preferably also has low substrate potential for Pgp or MRP1. More preferably, the active compound has low substrate potential for both Pgp and MRP1. Substrate potential for a transport protein can be determined by using an assay for measuring ATPase activity of the Pgp or MRP1 pumps (see, for example, Reference Example 4, below).

Methods for quantitating accumulation of the active compounds are readily available to one skilled in the art without undue experimentation (see U.S. Pat. No. 5,272,159 which is hereby incorporated by reference for the purpose of disclosing assays for quantitating accumulation). These assays use cell lines resistant to particular chemotherapeutic agents, and characterized by the presence of one or both of Pgp and MRP1. The cell line is exposed to a labeled form of the active compound (e.g., radioactivity or fluorescence labeling) and the accumulation of the active compound is monitored over time. The amount of active compound accumulated in the cell can be compared with a compound which is readily transported by these proteins, e.g. labeled TAXOL®.

### Compositions of this Invention

This invention further relates to a composition. The composition can be used for treating various conditions or disease states. The composition is preferably a pharmaceutical composition administered for treatment or prevention of multidrug resistance. Standard pharmaceutical formulation techniques are used, such as those disclosed in Remington's Pharmaceutical Sciences, Mack Publishing Company, Easton, Pa. (1990) and U.S. Pat. No. 5,091,187, which is hereby incorporated by reference.

The composition comprises component (A) the active compound described above and component (B) a carrier. The composition may further comprise component (C) an optional ingredient, such as a therapeutic agent.

Component (B) is a carrier. A carrier is one or more compatible substances that are suitable for administration to a mammal. “Compatible” means that the components of the composition are capable of being commingled with component (A), and with each other, in a manner such that there is no interaction which would substantially reduce the efficacy of the composition under ordinary use situations. Carriers must be of sufficiently high purity and sufficiently low toxicity to render them suitable for administration to the mammal being treated. The carrier can be inert, or it can possess pharmaceutical benefits, cosmetic benefits, or both, depending on the intended use as described herein.

The choice of carrier for component (B) depends on the route by which component (A) will be administered and the form of the composition. The composition may be in a variety of forms, suitable, for example, for systemic administration (e.g., oral, rectal, nasal, sublingual, buccal, or parenteral) or topical administration (e.g., local application on the skin, ocular, liposome delivery systems, or iontophoresis).

### Systemic Compositions

Carriers for systemic administration typically comprise one or more ingredients selected from the group consisting of a) diluents, b) lubricants, c) binders, d) disintegrants, e) colorants, f) flavors, g) sweeteners, h) antioxidants, j) preservatives, k) glidants, m) solvents, n) suspending agents, o) surfactants, combinations thereof, and others.

Ingredient a) is a diluent. Suitable diluents include sugars such as glucose, lactose, dextrose, and sucrose; polyols such as propylene glycol; calcium carbonate; sodium carbonate; glycerin; mannitol; sorbitol; and maltodextrin. The amount of ingredient a) in the composition is typically about 1 to about 99%.

Ingredient b) is a lubricant. Suitable lubricants are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil, and oil of theobroma. The amount of ingredient b) in the composition is typically about 1 to about 99%.

Ingredient c) is a binder. Suitable binders include polyvinylpyrrolidone; magnesium aluminum silicate; starches such as corn starch and potato starch; gelatin; tragacanth; and cellulose and its derivatives, such as sodium carboxymethylcellulose, ethylcellulose, methylcellulose, microcrystalline cellulose, and hydroxypropylmethylcellulose; carbomer; providone; acacia; guar gum; and xanthan gum. The amount of ingredient c) in the composition is typically about 1 to about 99%.

Ingredient d) is a disintegrant. Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of ingredient d) in the composition is typically about 1 to about 99%.

Ingredient e) is a colorant such as an FD&C dye. The amount of ingredient e) in the composition is typically about 1 to about 99%.

Ingredient f) is a flavor such as menthol, peppermint, and fruit flavors. The amount of ingredient f) in the composition is typically about 1 to about 99%.

Ingredient g) is a sweetener such as saccharin and aspartame. The amount of ingredient g) in the composition is typically about 1 to about 99%.

Ingredient h) is an antioxidant such as butylated hydroxyanisole, butylated hydroxytoluene, and vitamin E. The amount of ingredient h) in the composition is typically about 1 to about 99%.

Ingredient j) is a preservative such as phenol, alkyl esters of parahydroxybenzoic acid, benzoic acid and the salts thereof, boric acid and the salts thereof, sorbic acid and the salts thereof, chorbutanol, benzyl alcohol, thimerosal, phenylmercuric acetate and nitrate, nitromersol, benzalkonium chloride, cetylpyridinium chloride, methyl paraben, ethyl paraben, and propyl paraben. Particularly preferred are the salts of benzoic acid, cetylpyridinium chloride, methyl paraben and propyl paraben, and sodium benzoate. The amount of ingredient j) in the composition is typically about 1 to about 99%.

Ingredient k) is a glidant such as silicon dioxide. The amount of ingredient k) in the composition is typically about 1 to about 99%.

Ingredient m) is a solvent, such as water, isotonic saline, ethyl oleate, alcohols such as ethanol, glycerin, cremaphor, glycols (e.g., polypropylene glycol and polyethylene glycol), and buffer solutions (e.g., phosphate, potassium acetate, boric carbonic, phosphoric, succinic, malic, tartaric, citric, acetic, benzoic, lactic, glyceric, gluconic, glutaric, and glutamic). The amount of ingredient m) in the composition is typically about 1 to about 99%.

Ingredient n) is a suspending agent. Suitable suspending agents include AVICEL® RC-591 from FMC Corporation of Philadelphia, Pa. and sodium alginate. The amount of ingredient n) in the composition is typically about 1 to about 99%

Ingredient o) is a surfactant such as lecithin, polysorbate 80, sodium lauryl sulfate, polyoxyethylene sorbitan fatty acid esters, polyoxyethylene monoalkyl ethers, sucrose monoesters, lanolin esters, and lanolin ethers. Suitable surfactants are known in the art and commercially available, e.g., the TWEENS® from Atlas Powder Company of Wilmington, Del. Suitable surfactants are disclosed in the C.T.F.A. Cosmetic Ingredient Handbook, pp.587-592 (1992); Remington's Pharmaceutical Sciences, 15th Ed., pp. 335-337 (1975); and McCutcheon's Volume 1 Emulsifiers & Detergents, North American Edition, pp. 236-239 (1994). The amount of ingredient o) in the composition is typically about 1 to about 99%.

The carrier ingredients discussed above are exemplary and not limiting. One skilled in the art would recognize that different carrier ingredients may be added to or substituted for the carrier ingredients above. One skilled in the art would be able to select appropriate carrier ingredients for systemic compositions without undue experimentation.

Compositions for parenteral administration typically comprise (A) about 0.1 to about 10% of an active compound and (B) about 90 to about 99.9% of a carrier comprising a) a diluent and m) a solvent. Preferably, component a) is propylene glycol and m) is selected from the group consisting of ethanol, ethyl oleate, water, isotonic saline, and combinations thereof.

Compositions for oral administration can have various dosage forms. For example, solid forms include tablets, capsules, granules, and bulk powders. These oral dosage forms comprise a safe and effective amount, usually at least about 1%, and preferably from about 5% to about 50%, of component (A). The oral dosage compositions further comprise (B) about 50 to about 99% of a carrier, preferably about 50 to about 95%.

Tablets can be compressed, tablet triturates, enteric-coated, sugar-coated, film-coated, or multiple-compressed. Tablets typically comprise (A) the active compound, and (B) a carrier comprising ingredients selected from the group consisting of a) diluents, b) lubricants, c) binders, d) disintegrants, e) colorants, f) flavors, g) sweeteners, k) glidants, and combinations thereof. Preferred diluents include calcium carbonate, sodium carbonate, mannitol, lactose, and sucrose. Preferred binders include starch, and gelatin. Preferred disintegrants include alginic acid, and croscarmelose. Preferred lubricants include magnesium stearate, stearic acid, and talc. Preferred colorants are the FD&C dyes, which can be added for appearance. Chewable tablets preferably contain g) sweeteners such as aspartame and saccharin or f) flavors such as menthol, peppermint, and fruit flavors, or both.

Capsules (including time release and sustained release compositions) typically comprise (A) the active compound and (B) the carrier comprising one or more a) diluents disclosed above in a capsule comprising gelatin. Granules typically comprise (A) the active compound, and preferably further comprise k) glidants such as silicon dioxide to improve flow characteristics.

The selection of ingredients in the carrier for oral compositions depends on secondary considerations like taste, cost, and shelf stability, which are not critical for the purposes of this invention. One skilled in the art can optimize appropriate ingredients without undue experimentation.

The solid compositions may also be coated by conventional methods, typically with pH or time-dependent coatings, such that component (A) is released in the gastrointestinal tract at various times to extend the desired action. The coatings typically comprise one or more components selected from the group consisting of cellulose acetate phthalate, polyvinylacetate phthalate, hydroxypropyl methyl cellulose phthalate, ethyl cellulose, acrylic resins such as EUDRAGIT® coatings (available from Rohm & Haas G.M.B.H. of Darmstadt, Germany), waxes, shellac, polyvinylpyrrolidone, and other commercially available film-coating preparations such as Dri-Klear, manufactured by Crompton & Knowles Corp., Mahwah, N.J. or OPADRY® manufactured by Colorcon, Inc., of West Point, Pa.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise (A) the active compound and (B) a carrier comprising ingredients selected from the group consisting of a) diluents, e) colorants, and f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the active compounds include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethylcellulose, and hydroxypropylmethylcellulose. Such compositions may further comprise b) lubricants, e) colorants, f) flavors, g) sweeteners, h) antioxidants, and k) glidants.

The composition may further comprise component (C) one or more optional ingredients. Component (C) can be a therapeutic agent used to treat the underlying disease from which the subject suffers. For example, component (C) can be (i) a cancer therapeutic agent, such as a chemotherapeutic agent or a chemosensitizing agent, or a combination thereof; (ii) an antibacterial agent, (iii) an antiviral agent, (iv) an antifungal agent, and combinations thereof. Component (C) can be coadministered with component (A) to increase the susceptibility of the multidrug resistant cells within the subject to the therapeutic agent.

Suitable (i) cancer therapeutic agents are known in the art. Cancer therapeutic agents include chemotherapeutic agents, chemosensitizing agents, and combinations thereof. Suitable chemotherapeutic agents are disclosed in U.S. Pat. No. 5,416,091, which is hereby incorporated by reference for the purpose of disclosing chemotherapeutic agents. Suitable chemotherapeutic agents include actinomycin D, adriyamycin, amsacrine, colchicine, daunorubicin, docetaxel (which is commercially available as TAXOTERE® from Aventis Pharmaceuticals Products, Inc.), doxorubicin, etoposide, mitoxantrone, mytomycin C, paclitaxel (which is commercially available as TAXOL® from Bristol-Myers Squibb Company of New York, N.Y.), tenipaside, vinblastine, vincristine, and combinations thereof.

Suitable chemosensitizing agents include calcium channel blockers, calmodulin antagonists, cyclic peptides, cyclosporins and their analogs, phenothiazines, quinidine, reserpine, steroids, thioxantheres, transflupentixol, trifluoperazine, and combinations thereof. Suitable chemosensitizing agents are disclosed by Amudkar, et. al in “Biochemical, Cellular, and Pharmacological Aspects of the Multidrug Transporter,” Annu. Rev. Pharmacol. Toxicol., 39, pp. 361-398 (1999).

Suitable (ii) antibacterial agents, (iii) antiviral agents, and (iv) antifungal agents are known in the art (see “Annual Reports on Medicinal Chemistry—33; Section III Cancer and Infectious Diseases” ed. Plattner, J., Academic Press, Ch. 12, pp. 121-130 (1998)). Suitable antibacterial agents include quinolones, fluoroquinolones, β-lactam antibiotics, aminoglycosides, macrolides, glycopeptides, tetracyclines, and combinations thereof.

Suitable (iii) antiviral agents include protease inhibitors, DNA synthase inhibitors, reverse transcription inhibitors, and combinations thereof.

Suitable (iv) antifungal agents include azoles, such as ketoconazole, fluconazole, itraconazole, and combinations thereof.

One skilled in the art will recognize that these therapeutic agents are exemplary and not limiting, and that some may be used in the treatment of various multidrug resistant conditions and diseases. One skilled in the art would be able to select therapeutic agents without undue experimentation.

The amount of component (C) used in combination with component (A), whether included in the same composition or separately coadministered, will be less than or equal to that used in a monotherapy. Preferably, the amount of component (C) is less than 80% of the dosage used in a monotherapy. Monotherapeutic dosages of such agents are known in the art.

Component (C) may be part of a single pharmaceutical composition or may be separately administered at a time before, during, or after administration of component (A), or combinations thereof.

In a preferred embodiment, the composition of this invention comprises component (A), component (B), and (C) a chemotherapeutic agent. In an alternative preferred embodiment, the composition comprises component (A), component (B), and (C) a chemosensitizing agent. In another preferred alternative embodiment, the composition comprises component (A), component (B), and (C) both a chemotherapeutic agent and a chemosensitizing agent.

The exact amounts of each component in the systemic compositions depend on various factors. These factors include the specific compound selected as component (A). and the mode by which the composition will be administered. The amount of component (A) in the systemic composition is typically about 1 to about 99%.

The systemic composition preferably further comprises 0 to 99% component (C), and a sufficient amount of component (B) such that the amounts of components (A), (B), and (C), combined equal 100%. The amount of (B) the carrier employed in conjunction with component (A) is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

### Topical Compositions

Topical compositions comprise: component (A), described above, and component (B) a carrier. The carrier of the topical composition preferably aids penetration of component (A) into the skin. Topical compositions preferably further comprise (C) the optional ingredient described above.

Component (B) the carrier may comprise a single ingredient or a combination of two or more ingredients. In the topical compositions, component (B) is a topical carrier. Preferred topical carriers comprise one or more ingredients selected from the group consisting of water, alcohols, aloe vera gel, allantoin, glycerin, vitamin A and E oils, mineral oil, propylene glycol, polypropylene glycol-2 myristyl propionate, dimethyl isosorbide, combinations thereof, and the like. More preferred carriers include propylene glycol, dimethyl isosorbide, and water.

The topical carrier may comprise one or more ingredients selected from the group consisting of q) emollients, r) propellants, s) solvents, t) humectants, u) thickeners, v) powders, and w) fragrances in addition to, or instead of, the preferred topical carrier ingredients listed above. One skilled in the art would be able to optimize carrier ingredients for the topical compositions without undue experimentation.

Ingredient q) is an emollient. The amount of ingredient q) in the topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petrolatum, mineral oil, butyl myristate, isostearic acid, palmitic acid, isopropyl linoleate, lauryl lactate, myristyl lactate, decyl oleate, myristyl myristate, polydimethylsiloxane, and combinations thereof. Preferred emollients include stearyl alcohol and polydimethylsiloxane.

Ingredient r) is a propellant. The amount of ingredient r) in the topical composition is typically about 5 to about 95%. Suitable propellants include propane, butane, isobutane, dimethyl ether, carbon dioxide, nitrous oxide, nitrogen, and combinations thereof.

Ingredient s) is a solvent. The amount of ingredient s) in the topical composition is typically about 5 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Preferred solvents include ethyl alcohol.

Ingredient t) is a humectant. The amount of ingredient t) in the topical composition is typically about 5 to about 95%. Suitable humectants include glycerin, sorbitol, sodium 2-pyrrolidone-5-carboxylate, soluble collagen, dibutyl phthalate, gelatin, and combinations thereof. Preferred humectants include glycerin.

Ingredient u) is a thickener. The amount of ingredient u) in the topical composition is typically 0 to about 95%.

Ingredient v) is a powder. The amount of ingredient v) in the topical composition is typically 0 to about 95%. Suitable powders include chalk, talc, fullers earth, kaolin, starch, gums, colloidal silicon dioxide, sodium polyacrylate, tetraalkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

Ingredient w) is a fragrance. The amount of ingredient w) in the topical composition is typically about 0.001 to about 0.5%, preferably about 0.001 to about 0.1%.

Ingredient x) is a wax. Waxes useful in this invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point between 40 and 100° C. The amount of ingredient x) in the topical composition is typically about 1 to about 99%.

In an alternative embodiment of the invention, the active compounds may also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines. A preferred composition for topical delivery of the present compounds uses liposomes as described in Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Wallach and Philippot, “New Type of Lipid Vesicle: Novasome®”, Liposome Technology, Vol. 1, pp. 141-156 (1993); U.S. Pat. No. 4,911,928, and U.S. Pat. No. 5,834,014.

The exact amounts of each component in the topical composition depend on various factors. Including the specific compound selected for component (A) and the mode by which the composition will be administered. However, the amount of component (A) typically added to the topical composition is about 0.1 to about 99%, preferably about 1 to about 10%.

The topical composition preferably further comprises 0 to about 99% component (C), more preferably 0 to abut 10%, and a sufficient amount of component (B) such that the amounts of components (A), (B), and (C), combined equal 100%. The amount of (B) the carrier employed in conjunction with component (A) is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Topical compositions that can be applied locally to the skin may be in any form including solutions, oils, creams, ointments, gels, lotions, shampoos, leave-on and rinse-out hair conditioners, milks, cleansers, moisturizers, sprays, skin patches, and the like.

Component (A) may be included in kits comprising component (A), a systemic or topical composition described above, or both; and information, instructions, or both that use of the kit will provide treatment for multidrug resistance (particularly in humans). The information and instructions may be in the form of words, pictures, or both, and the like. In addition or in the alternative, the kit may comprise component (A), a composition, or both; and information, instructions, or both, regarding methods of administration of component (A) or the composition, preferably with the benefit of treating multidrug resistance in mammals.

In an alternative embodiment of the invention, components (A) and (C) may be included in kits comprising components (A) and (C), systemic or topical compositions described above, or both; and information, instructions, or both that use of the kit will provide treatment for multidrug resistance (particularly humans). The information and instructions may be in the form of words, pictures, or both, and the like. In addition or in the alternative, the kit may comprise components (A) and (C), compositions, or both; and information, instructions, or both, regarding methods of administration of components (A) and (C) or the compositions, preferably with the benefit of treating multidrug resistance in mammals.

### Methods of Use of the Invention

This invention relates to a method of inhibiting a transport protein. The method comprises administering to a mammal in need of treatment, (A) an active compound described above.

This invention further relates to a method for treating multidrug resistance. The method comprises administering to a mammal (preferably a human) suffering from multidrug resistance, (A) an active compound described above. For example, a mammal diagnosed with multidrug resistant cancer can be treated by the methods of this invention. Preferably, a systemic or topical composition comprising (A) the active compound and (B) the carrier is administered to the mammal. More preferably, the composition is a systemic composition comprising (A) the active compound, (B) the carrier, and (C) an optional ingredient such as a therapeutic agent. Component (A) may be administered before, during, or after administration of component (C). A preferred administration schedule is a continuous infusion over the 24 hour period during which component (C) is also administered.

The dosage of component (A) administered depends on various factors, including the method of administration, the physical attributes of the subject (e.g., age, weight, and gender), and the condition from which the subject suffers. Effective dosage levels for treating or preventing MDR range from about 0.01 to about 100 mg/kg body weight per day, preferably about 0.5 to about 50 mg/kg body weight per day of (A) a compound of this invention. These dosage ranges are merely exemplary, and daily administration can be adjusted depending on various factors. The specific dosage of the active compound to be administered, as well as the duration of treatment, and whether the treatment is topical or systemic are interdependent. The dosage and treatment regimen will also depend upon such factors as the specific active compound used, the treatment indication, the efficacy of the active compound, the personal attributes of the subject (such as, for example, weight, age, sex, and medical condition of the subject), compliance with the treatment regimen, and the presence and severity of any side effects of the treatment.

In addition to the benefits in treating multidrug resistance in subjects suffering from cancer, the active compounds in the compositions and methods of this invention can also be used to treat other conditions. These other conditions include other types of multidrug resistance (i.e., in addition to cancer multidrug resistance) such as bacterial, viral, and fungal multidrug resistance. For example, many of the FDA approved HIV protease inhibitors used to treat AIDS patients suffering from the HIV virus are substrates for Pgp. Therefore, in an alternative embodiment of this invention, an active compound of this invention is coadministered with a therapeutic agent such as an HIV protease inhibitor.

The active compounds and compositions of this invention can also be administered with other therapeutic agents such as oral drugs. The active compounds and compositions can be used to enhance oral drug absorption and increase bioavailability of various drugs.

The active compounds and compositions can also be used to aid drug delivery through the blood-brain barrier for, e.g., enhancing the effectiveness of drugs to treat Alzheimer's disease, treating memory disorders, enhancing memory performance, or treating any other central nervous system disorder where drug delivery is compromised via this transport pump mechanism.

The active compounds and compositions can also be administered to treat subjects suffering from neurological disorders such as spinal injuries, diabetic neuropathy, and macular degeneration.

The active compounds and compositions can also be administered to treat subjects suffering from vision disorders and to improve vision.

The active compounds and compositions can also be administered to treat hair loss. “Treating hair loss” includes arresting hair loss, reversing hair loss, and promoting hair growth.

The active compounds and compositions can also be adminstered to treat inflammatory diseases. Inflammatory diseases include irritable bowel disease, arthritis, and asthma.

### EXAMPLES

These examples are intended to illustrate the invention to those skilled in the art and should not be interpreted as limiting the scope of the invention set forth in the claims. The active compounds of this invention can be made using conventional organic syntheses, which are readily available to one skilled in the art without undue experimentation. Such syntheses can be found in standard texts such as J. March, Advanced Organic Chemistry, John Wiley & Sons, 1992. One of ordinary skill in the art will appreciate that certain reactions are best carried out when other functionalities are masked or protected in the compound, thus increasing the yield of the reaction or avoiding any undesirable side reactions. The skilled artisan may use protecting groups to accomplish the increased yields or to avoid the undesired reactions. These reactions can be found in the literature, see for example, Greene, T. W. and Wuts, P. G. M., Protecting Groups in Organic Synthesis, 2^{nd} ed., John Wiley & Sons, 1991.

The starting materials for preparing the compounds of the invention are known, made by known methods, or commercially available. The starting materials for preparing the compounds of the invention may include the following.

The following reagents are available from Aldrich Chemical Company, Milwaukee, Wis.: 1-bromo-3-phenylpropane, 5-hydroxyquinoline, (R)-(−)-glycidyl tosylate, 3,4-pyridinedicarboxylic acid, 4-phenylbutylamine, 3-pyridinepropionic acid, tert-butyl[S-(R*, R*)]-(−)-(1-oxiranyl)-2-phenylethyl)carbamate, epichlorohydrin, 3,4,5-trimethoxybenzoyl chloride, N,N-diisopropylethylamine, 4-dimethylaminopyridine, 1-hydroxybenzotriazole, 4-trans-aminomethylcyclohexanecarboxylic acid, 3,4,5-trimethoxybenzylamine, and 2,2,4-trimethyl-2-oxazoline.

The following reagents are available from Lancaster Synthesis Inc., Windham, N.H.: 4-phenylbutyronitrile, 1-tert-butoxycarbonyl-piperidine-3-carboxylic acid, 1-benzyl-4-aminopiperidine, 3,4-dimethoxybenzenesulfonyl chloride, 1-(carboxymethyl)piperazine ethyl ester, and 1-benzyl-4-homopiperazine.

The following reagents are available from Fluka Chemie AG, Milwaukee, Wis.: 1-tert-butoxycarbonyl-piperidine-4-carboxylic, and (benzotriazol-1-yloxy)tripyrrolidinophosphonium hexafluorophosphate (“PyBOP”), N-(tert-butoxycarbonyl)-iminodiacetic acid, and 1-(diphenylmethyl)piperazine.

The following reagents are available from Acros Organics, Pittsburgh, Pa: quinoline-6-carboxylic acid and quinoline-5-carboxylic acid.

The following reagent is available from Bachem Bioscience, King of Prussia, Pa.: tert-butoxycarbonyl-β-(3-pyridyl)-alanine.

The following reagent is available from Sigma Chemical Company, Milwaukee, Wis.: N-(3-dimethylaminopropyl)-N′-ethylcarbodiimide hydrochloride.

Various abbreviations are used herein. Abbreviations that can be used and their definitions are shown below in Table 2.

TABLE 2
Abbreviations

| Abbreviation | Definition |
|---|---|
| “AM” | acetoxymethyl ester |
| “Boc” | tert-butoxycarbonyl |
| “CIMS” | chemical ionization mass spectrometry |
| “DMF” | dimethylformamide |
| “ESMS” | electrospray mass spectrometry |
| “Et” | an ethyl group |
| “ISMS” | ion-spray mass spectrometry |
| “Me” | a methyl group |
| “MH+” | parent ion in ESMS |
| “MS” | mass spectrometry |
| “MTT” | 3-[4,5-dimethyl-thiazoyl-2-yl]2,5-diphenyl- |
| | tetrazolium bromide |
| “NIH” | National Institute of Health |
| “PBS” | Phosphate-buffered saline |
| “THF” | tetrahydrofuran |

### Reference Example 1

### Method for Measuring Activity to Inhibit Pgp (Reversal Assay)

NIH-MDR1-G185 cells (obtained from M. Gottesman, NIH) were harvested and resuspended at 6×10⁴ cells/ml in RPMI 1640 containing L-glutamine, 10% Cosmic calf serum, and penicillin-streptomycin. Cell suspension aliquots of 100 microliters were added to individual wells of a 96 well microtiter plate and incubated overnight at 37° C. to allow cells to adhere. Cell viability in the presence of an anticancer drug was determined in the presence and absence of an MDR modifying agent using an MTT assay (P. A. Nelson, et. al, J. Immunol, 150:2139-2147 (1993)).

Briefly, cells were preincubated with an MDR modulating agent (final concentration 5 micromolar) for 15 min at 37° C., then treated with varying concentrations of an anticancer agent for 72 hr at 37° C. MTT dye (20 microliters of 5 mg/ml PBS solution) was added to each well and incubated for 4 hr at 37° C. Media was carefully removed and dye was solubilized with 100 microliters of acidified isopropyl alcohol. Absorption was measured on a spectrophotometric plate reader at 570 nm and corrected for background by subtraction at 630 nm. Reversal index was calculated for each MDR modulator and normalized to the reversal index of a benchmark modulator, VX-710 as below:

Reversal index =IC₅₀ in the absence of modulator/IC₅₀ in the presence of modulator

Normalized reversal index =Reversal index of modulator/Reversal index of VX-710

VX-710 is (S)-N-[2-Oxo-2-(3 ,4,5-trimethoxyphenyl)acetyl]piperidine-2-carboxylic acid 1,7-bis(3-pyridyl)-4-heptyl ester.

### Reference Example 2

### Method for Measuring Activity to Inhibit Pgp and MRP1 (Calcein AM Extrusion Assay)

Pgp-dependent calcein AM extrusion was measured in NIH-MDR1-G185 cells or HL60-MDR1 cells. MRP1-dependent calcein AM extrusion was measured in HL60/ADR cells. Dye uptake was measured by incubating 0.5-1×10⁶ cells/ml in cell culture medium containing 0.25 mM calcein AM at 37° C. at an excitation wavelength=493 nm and an emission wavelength=515 nm. Inhibition of calcein AM transport by varying concentrations of MDR modulators was determined by measuring the rate of increase in fluorescence of free calcein for 5 min periods. The IC₅₀ values were obtained by determining the concentration of modulator resulting in 50% of the maximum transport inhibition. Maximum transport inhibition was the % inhibition produced in the presence of 50-60 micromolar verapmil.

### Reference Example 3

### Fluorescent Substrate Accumulation Assay

NIH-MDR1-G185 cells (obtained from M. Gottesman, NIH) were harvested and resuspended in RPMI-1640 containing L-glutamine, 10% Cosmic Calf Serum and penicillin-streptomycin. Cell suspension aliquots of 175 microliters (1×10⁵ cells) were added to individual wells of a 96 well microtiter plate and preincubated for 15 min at 37° C. with 20 microliters MDR modulator diluted in cell culture media to give a final concentration of 10 micromolar. Control wells received no modulating agent. BODIPY-FL Taxol (Molecular Probes, Eugene, Oreg.) was added to each well in 10 microliter aliquots to give a final concentration of 500 nM and cells were incubated for 40 min at 37° C. Cells were centrifuged at 100×g for 5 min at 4° C. and the cell pellet washed with 200 microliters cold PBS to remove fluorescent medium from wells. Cells were centrifuged once more, media removed, and cells resuspended in 200 microliters cold PBS. Fluorescence accumulation was measured in a fluorescence plate reader fitted with an excitation filter of 485 nm and an emission filter of 538 nm. BODIPY-FL taxol accumulation in the cells was calculated as follows:

Accumulation Index=(fluorescence in NIH-MDR1-G185 cells in the presence of modulator)/(fluorescence in NIH-MDR1-G185 cells in absence of modulator)

### Reference Example 4

### Method for Measuring Substrate Potential for MDR1(MDR1 ATPase assay)

Recombinant baculovirus carrying the human MDR1 gene was generated and Sf9) cells infected with virus. The virus-infected cells were harvested and their membranes isolated. MDR1-ATPase activity of the isolated Sf9 cell membranes was estimated by measuring inorganic phosphate liberation as previously described (B. Sarkadi, J. Biol. Chem., 1992, 267:4854-4858). The differences between the ATPase activities measured in the absence and presence of 100 micromolar vanadate were determined as activity specific to MDR1. MDR modulator concentrations causing half-maximum activation (Ka) or half-maximum inhibition of the MDR1-ATPase stimulated by 30-40 micromolar verapamil (Ki) were determined.

### Example 1

### Preparation of 5-Phenyl-2-(3-phenyl-propyl)-pentanoic acid (1)

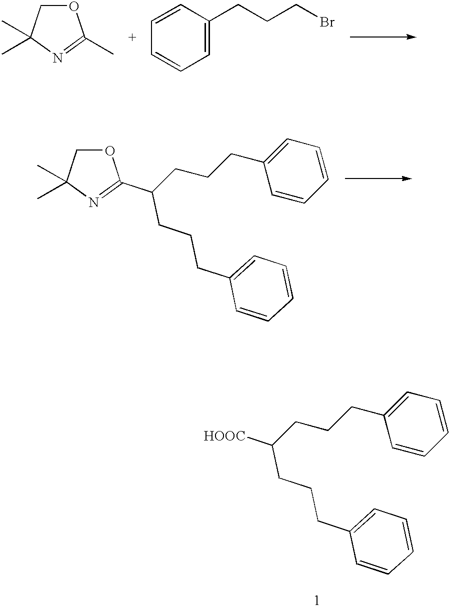

2,2,4-Trimethyl-2-oxazoline (5.64 mL; 44.2 mmol) is dissolved in THF (40 ml in a dry, argon purged flask at ambient temperature. The solution is cooled to −78° C., then n-butyllithium in hexanes (31.3 mL of 1.6 M solution; 50 mmol) is added dropwise via syringe, followed by a solution of 1-bromo-3-phenylpropane (7.42 mL; 48.8 mmol) in THF (20 mL) dropwise via syringe. The cooling bath is removed and the solution is allowed to slowly warm to ambient temperature. After approximately 30 minutes, the reaction is cooled to −78° C., then n-butyllithium in hexanes (31.3 mL of 1.6 M solution; 50 mmol) is added dropwise via syringe, followed by a solution of 1-bromo-3-phenylpropane (7.42 mL; 48.8 mmol) in THF (20 mL) dropwise via syringe. The reaction mixture is stirred overnight with very slow warming to ambient temperature. The solution is poured onto water (200 mL) and 1N HCl is added to make the mixture acidic. The mixture is extracted with ether (150 mL), then made alkaline with 50% aqueous sodium hydroxide solution. The alkaline mixture is extracted with ether (3×100 mL). The combined ether extracts are dried over MgSO₄, filtered, and concentrated in vacuo. The residue is purified via silica gel chromatography with gradient elution (0%→33% ethyl acetate in hexanes) affording the dialkylated oxazoline intermediate (13.55 g) as a colorless liquid. ESMS: MH⁺ 349.6 (base). The dialkylated oxazoline intermediate (1 g; 2.86 mmol) is dissolved in dioxane (10 mL) at ambient temperature. 3N HCl (20 mL) is added and the solution is heated to gentle reflux for 18 hours. After cooling, the reaction mixture is poured onto water (20 mL) and extracted with ether (3×30 mL). The combined ether extracts are washed successively with water (20 mL), and brine (20 mL), then dried over MgSO₄, filtered, and concentrated in vacuo at 40° C. affording the desired product (0.76g) as a solid. ESMS: MH⁺ 297.2.

### Example 2

### Preparation of (R)-5-oxiranylmethoxy-quinoline (2)

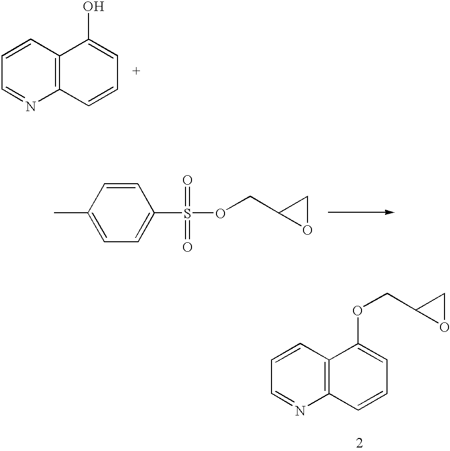

Sodium hydride (60 weight %; 1.79 g; 44.8 mmol) is washed with hexanes (3×10 mL) under an argon blanket. DMF (17 mL) is then added at ambient temperature and the stirred slurry is cooled to 5° C. A solution of 5-hydroxyquinoline (5.00 g; 34.4 mmol) in DMF (65 mL) is added dropwise over 30 minutes. The resulting mixture is allowed to warm to ambient temperature over 1 hour affording a clear, reddish-brown solution. A solution of (R)-(−)-glycidyl tosylate (10.22 g; 44.8 mmol) in DMF (50 mL) is added dropwise over 20 minutes. The resulting mixture is stirred at ambient temperature for 4 hours, quenched by the addition of saturated aqueous ammonium chloride (25 mL), poured onto water (750 mL), and extracted with ether (3×375 mL). The combined ether layers are washed with saturated aqueous sodium bicarbonate (2×375 mL), then dried over MgSO₄, filtered, and concentrated in vacuo. The residue is purified via silica gel chromatography with gradient elution (33%→50% ethyl acetate in hexanes) affording the desired product (4.95 g) as a tan solid. ESMS: MH⁺ 202.2 (base).

### Example 3

### Preparation of 1-tert-butoxycarbonyl-4-(4′-benzylpiperazine)piperidine (3)

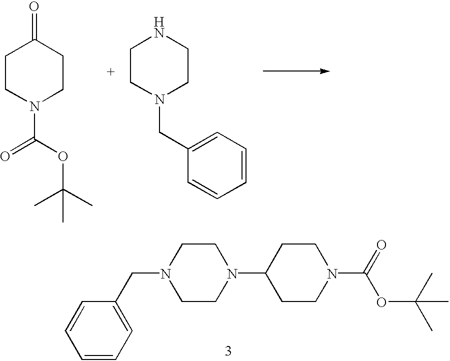

tert-Butyl 4-oxo-1-piperidinecarboxylate (5.19 g; 26 mmol) is dissolved in isopropanol (100 mL) at ambient temperature. 1-Benzylpiperazine (4.53 g; 22 mmol) is added followed by glacial acetic acid (4 mL; 7 mmol). Lastly, sodium cyanoborohydride (1.72 g; 27 mmol) is added portionwise over 15 minutes. The mixture is stirred for 18 hours at ambient temperature then concentrated in vacuo. The residue is slurried in ethyl acetate (200 mL), then extracted sequentially with water (100 mL), saturated aqueous sodium bicarbonate (50 mL), and brine (50 mL). The organic layer is dried over MgSO₄, filtered, and concentrated in vacuo. The residue is purified via silica gel chromatography with gradient elution (0%→50% acetone in hexanes) affording the desired product (7.15 g) as a colorless oil. ESMS: MH⁺ 360.4 (base).

### Example 4

### Preparation of 1-tert-butoxycarbonyl-4-(piperazine)piperidine (4)

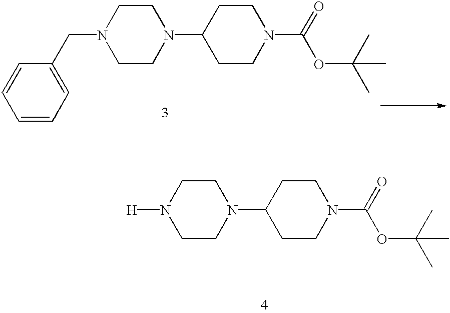

A mixture of 1-tert-butoxycarbonyl-4-(4′-benzylpiperazine)piperidine (3) (1.00 g; 2.28 mmol), ethanol (25 mL) and Pearlman's catalyst (Pd(OH)₂ on carbon; 1.2 g) is hydrogenated at 50 psi for 120 hours at ambient temperature. The mixture is filtered through a pad of diatomaceous earth and washed with ethanol. The filtrate is concentrated in vacuo affording the desired product (0.71 g) as an oil. ESMS: MH⁺ 270.2 (base).

### Example 5

### Preparation of 1-tert-butoxylcarbonyl 4-[5-Phenyl-2-(3-phenyl-propyl)-pentanoic acid piperazin-4-ylamide]piperidine (5)

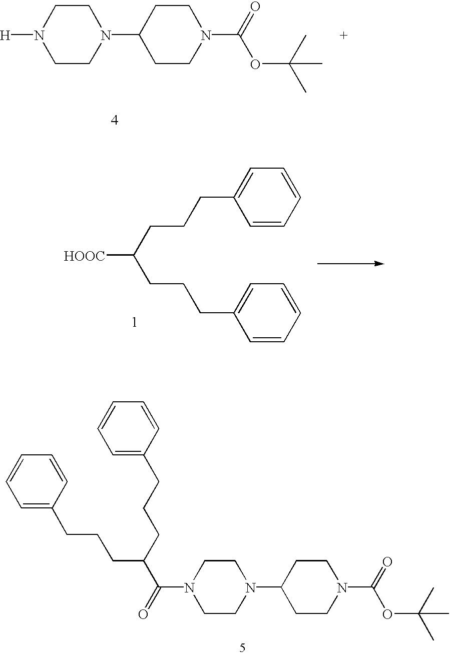

1-tert-Butoxycarbonyl-4-(piperazine)piperidine (4) (0.38 g; 1.41 mmol) is dissolved in DMF (10 mL) at ambient temperature. 5-Phenyl-2-(3-phenyl-propyl)-pentanoic acid (1) (0.42 g; 1.41 mmol), 1-hydroxybenzotriazole (0.24 g; 1.78 mmol), triethylamine (0.25; 1.79 mmol), and N-(3-dimethylaminopropyl)-N′-ethylcarbodiimide hydrochloride (0.30 g; 1.57 mmol) are added sequentially. The mixture is stirred for 18 hours at ambient temperature then poured onto ethyl acetate (150 mL) and extracted sequentially with water (50 mL), saturated NaHCO₃ (25 mL), and brine (25 mL). The organic layer is dried (MgSO₄), filtered, and concentrated in vacuo. The residue is purified via silica gel chromatography with gradient elution (0%→100% acetone in hexanes) affording the desired compound (0.2756 g) as a lightly colored liquid. ESMS: MH⁺ 548.6 (base).

### Example 6

### Preparation of 4-[5-phenyl-2-(3-phenyl-propyl)-pentanoic acid piperazin-4-ylamide]piperidine (6)

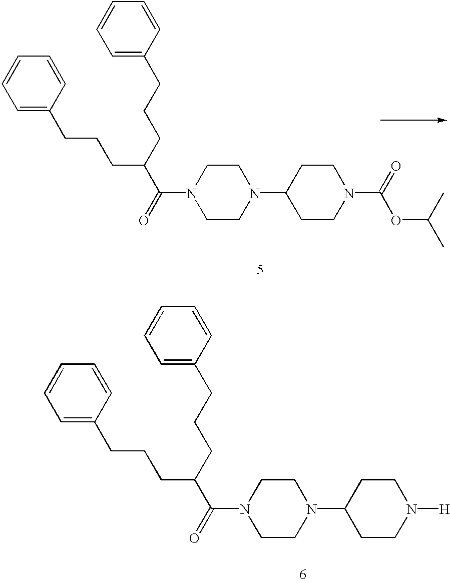

1-tert-Butoxylcarbonyl 4-[5-phenyl-2-(3-phenyl-propyl)-pentanoic acid piperazin-4-ylamide]piperidine (5) (0.2756 g; 0.50 mmol) is dissolved in methylene chloride (5 mL) at ambient temperature. Trifluoroacetic acid (5 mL) is added in a slow stream, and the solution is stirred for 90 minutes at ambient temperature. The solution is concentrated in vacuo. The residue is slurried in a mixture of methylene chloride (10 mL) and water (30 mL), then potassium carbonate is added until the slurry is alkaline. The slurry is diluted with water (20 mL) then extracted with methylene chloride (3×20 mL). The organic extracts are dried over MgSO₄, filtered, and concentrated in vacuo affording the desired product (0.2051 g) as a lightly colored oil. ESMS: MH⁺ 448.4 (base).

### Example 7

### Preparation of 4-[5-phenyl-2-(3-phenyl-propyl)-pentanoic acid {1-[2-hydroxy-3-(quinolin-5-yloxy)-propyl]-piperazin-4-yl}-amide]piperidine (7)

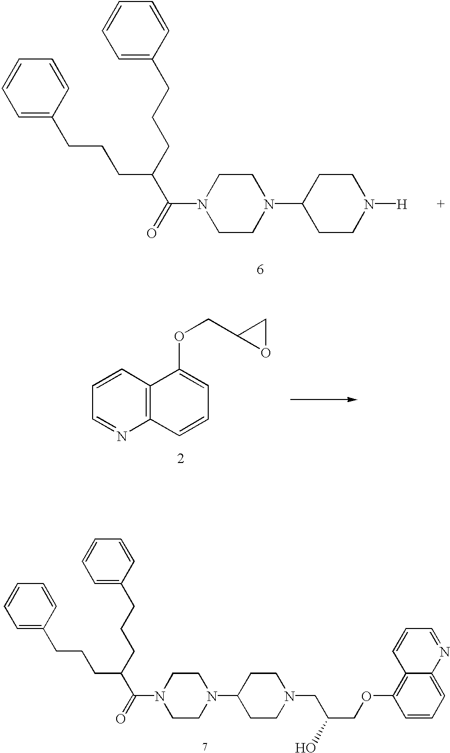

4-[5-Phenyl-2-(3-phenyl-propyl)-pentanoic acid piperazin-4-ylamide]piperidine (6) (0.2051 g; 0.46 mmol) is dissolved in isopropanol (10 mL) at ambient temperature. (R)-5-Oxiranylmethoxy-quinoline (2) (0.0923 g; 0.46 mmol) is added, then the mixture is heated to 70° C. and maintained for 18 hours. After cooling to ambient temperature, the solution is concentrated in vacuo. The residue is purified via silica gel chromatography with gradient elution (0%→50% methanol in methylene chloride) affording the desired product (0.1801 g) as a lightly colored oil. ESMS: MH⁺ 649.4 (base).

### Example 8

### Preparation of 1-[tert-butoxylcarbonyl-4-[2-hydroxy-3-(quinolin-5-yloxy)-propyl]-piperazin-4-yl}]piperidine (8)

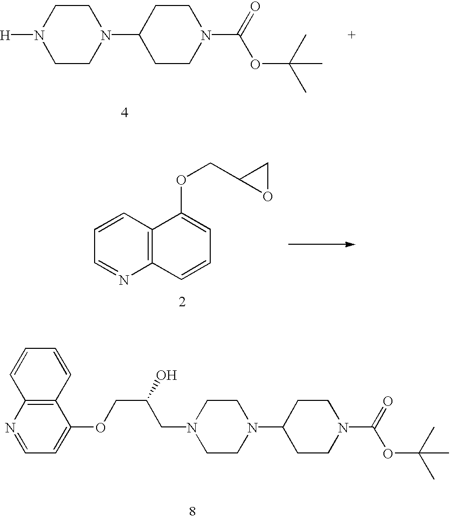

1-tert-Butoxycarbonyl-4-(1′-piperazine)piperidine (4) (0.4255 g; 1.58 mmol) is dissolved in isopropanol (20 mL) at ambient temperature. (R)-5-Oxiranylmethoxy-quinoline (2) (0.3178 g; 1.58 mmol) is added, then the mixture is heated to 70° C. and maintained for 18 hours. After cooling to ambient temperature, the solution is concentrated in vacuo. The residue is purified via silica gel chromatography with gradient elution (0%→50% methanol in methylene chloride) affording the desired product (0. 1624 g) as an amber colored oil. ESMS: MH⁺ 471.4 (base).

### Example 9

### Preparation of {4-[2-hydroxy-3-(quinolin-5-yloxy)-propyl]-piperazin-4-yl}piperidine (9)

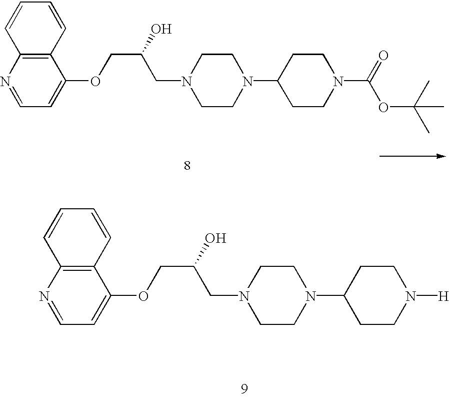

1[-tert-Butoxylcarbonyl {4-[2-hydroxy-3-(quinolin-5-yloxy)-propyl]-piperazin-4-yl}-amide]piperidine (8) (0.1624 g; 0.345 mmol) is dissolved in methylene chloride (7.5 mL) at ambient temperature. Trifluoroacetic acid (7.5 mL) is added in a slow stream, and the solution is stirred for 90 minutes at ambient temperature. The solution is concentrated in vacuo. The residue is slurried in a mixture of methylene chloride (10 mL) and water (30 mL), then potassium carbonate is added until the slurry is alkaline. The slurry is diluted with water (20 mL) then extracted with methylene chloride (3×20 mL). The organic extracts are dried over MgSO₄, filtered, and concentrated in vacuo affording the desired product (0.0697 g) as a yellow foam. ESMS: MH⁺ 371.2 (base).

### Example 10

### Preparation of 1-[5-Phenyl-2-(3-phenyl-propyl)-pentanoic acid {4-[2-hydroxy-3-(quinolin-5-yloxy)-propyl]-piperazin-4-yl}-amide]piperidine (10)

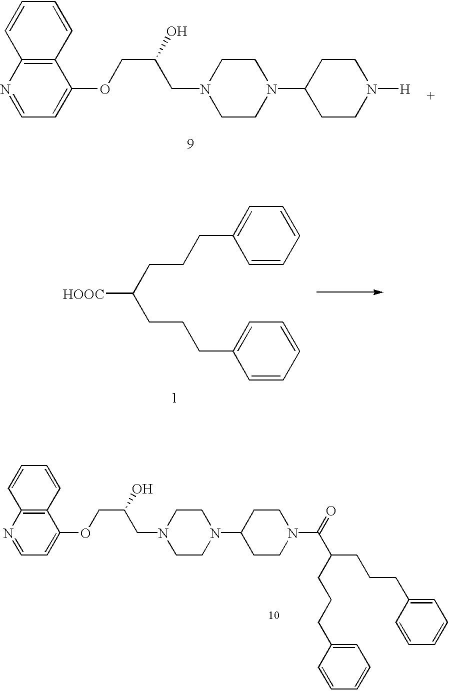

{4-[2-Hydroxy-3-(quinolin-5-yloxy)-propyl]-piperazin-4-yl}-amide piperidine (9) (0.0697 g; 0.188 mmol) is dissolved in DMF (5 mL) at ambient temperature. 5-Phenyl-2-(3-phenyl-propyl)-pentanoic acid (1) (0.0557 g; 0.188 mmol), 1-hydroxybenzotriazole (0.0318 g; 0.235 mmol), triethylamine (0.0524 mL; 0.376 mmol), and N-(3-dimethylaminopropyl)-N′-ethylcarbodiimide hydrochloride (0.0400 g; 0.209 mmol) are added sequentially. The mixture is stirred for 18 hours at ambient temperature then poured onto ethyl acetate (150 mL) and extracted sequentially with water (50 mL), saturated NaHCO₃ (25 mL), and brine (25 mL). The organic layer is dried (MgSO₄), filtered, and concentrated in vacuo. The residue is purified via silica gel chromatography with gradient elution (0%→50% methanol in methylene chloride) affording the desired compound (0.0937 g) as an oil. ESMS: MH⁺ 649.4.

### Example 11

### Preparation of 1-tert-Butoxycarbonyl-4-amino-(1′-benzyl-4′-piperidine)piperidine (11)

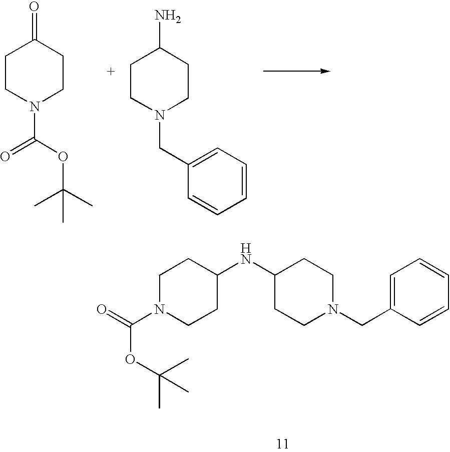

tert-Butyl 4-oxo-1-piperidinecarboxylate (0.25 g; 1.26 mmol) is dissolved in THF (10 mL) at ambient temperature. 4-Amino-1-benzylpiperidine (0.24 g; 1.26 mmol) is added followed by glacial acetic acid (0.1438 mL; 2.5 mmol). Lastly, sodium triacetoxyborohydride (0.293 g; 1.38 mmol) is added in one portion. The mixture is stirred for 18 hours at ambient temperature then concentrated in vacuo. The residue is slurried in ethyl acetate (100 mL), then extracted sequentially with saturated aqueous sodium bicarbonate (50 mL), and brine (50 mL). The organic layer is dried over MgSO₄, filtered, and concentrated in vacuo. The residue is purified via silica gel chromatography with gradient elution (0%→50% methanol in methylene chloride containing 0.1% NH₄OH) affording the desired product (0.29 g) as an oil. ESMS: MH⁺ 374.4 (base).

### Example 12

### Preparation of 1-tert-butoxycarbonyl-4-amino-(4′-piperidine)piperidine (12)

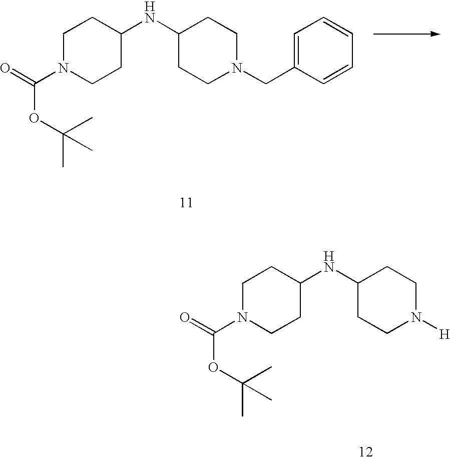

A mixture of 1-tert-butoxycarbonyl-4-amino-(1′-benzyl-4′-piperazine)piperidine (11) (0.29 g; 0.78 mmol), ethanol (25 mL) and Pearlman's catalyst (Pd(OH)₂ on carbon;

0.29 g) is hydrogenated at 50 psi for 24 hours at ambient temperature. The mixture is filtered through a pad of diatomaceous earth and washed with ethanol. The filtrate is concentrated in vacuo affording the desired product (0.2323 g) as an oil. ESMS: MH⁺ 284.2 (base).

### Example 13

### Preparation of 1-[tert-butoxylcarbonyl-4-amino-{(4′-piperidine)-1-[5-phenyl-2-(3-phenyl-propyl)-pentanoic acid]}piperidine (13)

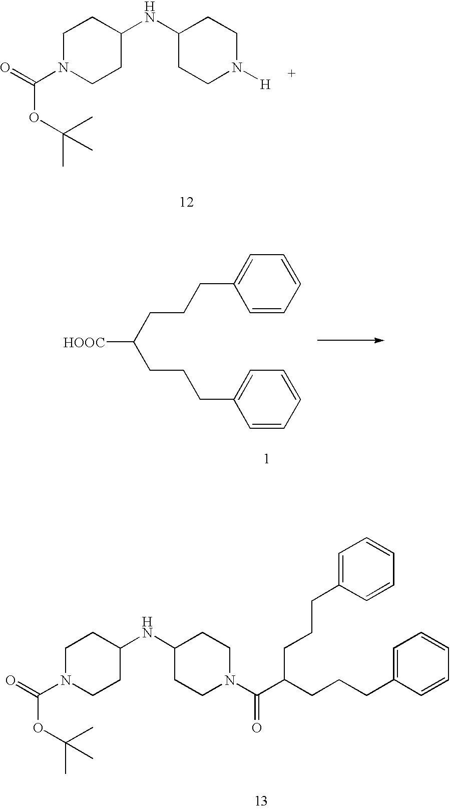

1-tert-Butoxycarbonyl-4-amino-(4′-piperidine)piperidine (12) (0.2200 g; 0.777 mmol) is dissolved in DMF (5 mL) at ambient temperature. 5-Phenyl-2-(3-phenyl-propyl)-pentanoic acid (1) (0.2304 g; 0.777 mmol), 1-hydroxybenzotriazole (0.1313 g; 0.972 mmol), triethylamine (0.2167 mL; 1.55 mmol), and N-(3-dimethylaminopropyl)-N′-ethylcarbodiimide hydrochloride (0.1640 g; 0.856 mmol) are added sequentially. The mixture is stirred for 18 hours at ambient temperature then poured onto ethyl acetate (150 mL) and extracted sequentially with water (50 mL), saturated NaHCO₃ (25 mL), and brine (25 mL). The organic layer is dried (MgSO₄), filtered, and concentrated in vacuo. The residue is purified via silica gel chromatography with gradient elution (10%→100% acetone in hexanes) affording the desired compound (0.3093 g) as an amber oil. ESMS: MH⁺ 562.4 (base).

### Example 14

### Preparation of 4-amino-{(4′-piperidine)-1-[5-phenyl-2-(3-phenyl-propyl)-pentanoic acid]}piperidine (14)

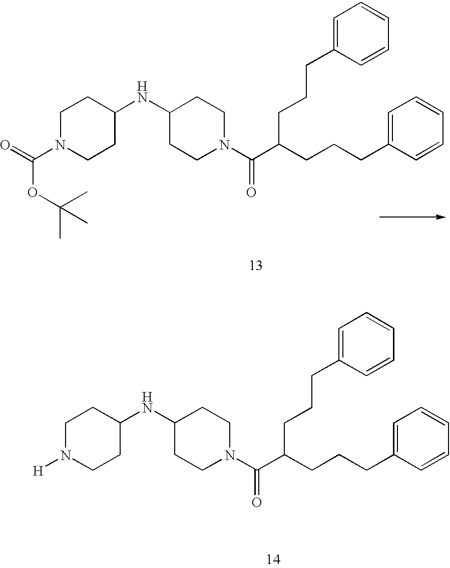

1-[tert-Butoxylcarbonyl-4-amino-{(4′-piperidine)1-[5-phenyl-2-(3-phenyl-propyl)-pentanoic acid]}piperidine (13)(0.31 g; 0.553 mmol) is dissolved in methylene chloride (15 mL) at ambient temperature. Trifluoroacetic acid (15 mL) is added in a slow stream, and the solution is stirred for 90 minutes at ambient temperature. The solution is concentrated in vacuo. The residue is slurried in a mixture of methylene chloride (10 ml,) and water (30 mL), then potassium carbonate is added until the slurry is alkaline. The slurry is diluted with water (20 mL) then extracted with methylene chloride (3×20 mL). The organic extracts are dried over MgSO₄, filtered, and concentrated in vacuo affording the desired product (0.2105 g) as an amber oil. ESMS: MH⁺ 462.4 (base).

### Example 15

### Preparation of 4-amino-{(4′-piperidine)-1-[5-Phenyl-2-(3-phenyl-propyl)-pentanoic acid]}{1-[2-hydroxy-3-(quinolin-5-yloxy)-propyl]-piperazin-4-yl}-amide]piperidine (15)

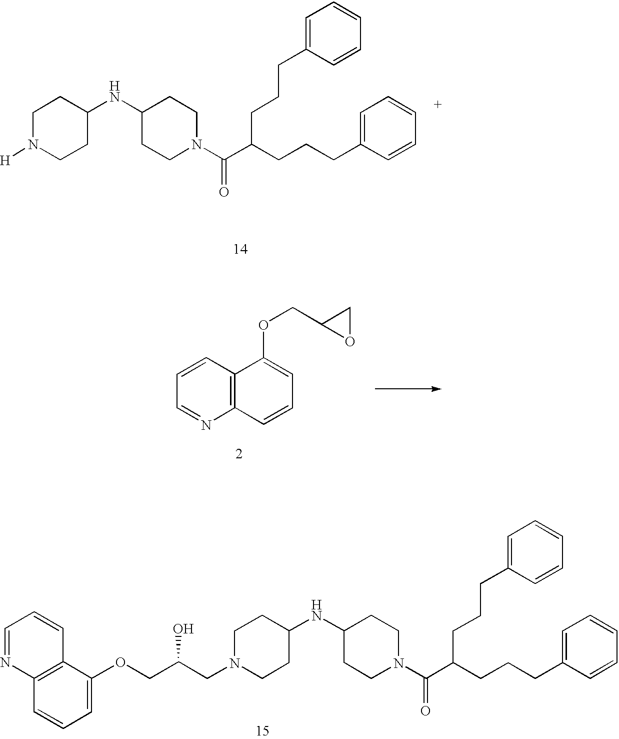

4-Amino-{(4′-piperidine)-[5-phenyl-2-(3-phenyl-propyl)-pentanoic acid]}piperidine (14) (0.2105 g; 0.457 mmol) is dissolved in isopropanol (10 mL) at ambient temperature. (R)-5-Oxiranylmethoxy-quinoline (2) (0.0918 g; 0.456 mmol) is added, then the mixture is heated to 70° C. and maintained for 18 hours. After cooling to ambient temperature, the solution is concentrated in vacuo. The residue is purified via silica gel chromatography with gradient elution (0%→50% methanol in methylene chloride) affording the desired product (0.2015 g) as a light yellow colored foam. ESMS: MH⁺ 663.6 (base).

### Example 16

### Preparation of N-(tert-butoxycarbonyl)-trans-4-(aminomethyl )-1-cyclohexanecarboxylic acid (16):

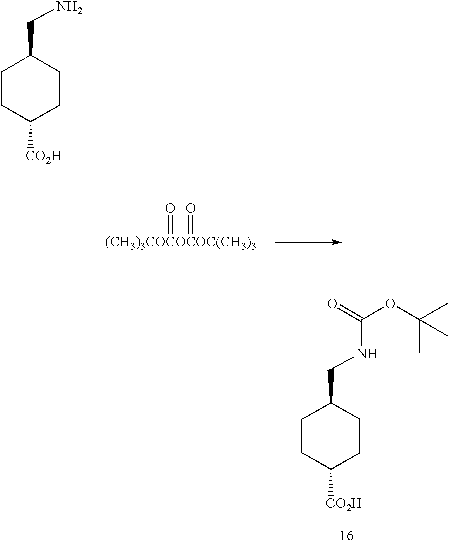

trans-4-(Aminomethyl)-1-cyclohexanecarboxylic acid (3.00 g, 19.1 mmol) is dissolved in 1N sodium hydroxide (20.8 mL, 20.8 mmol) and tert-butanol (25 mL). Di-tert-butyl dicarbonate (4.16 g, 19.1 mmol) is added and the reaction stirred for 4 hours at ambient temperature. The mixture is washed with hexane (3×30 mL). The aqueous phase is treated with ice-cold 1N HCl (30 mL) and extracted with ethyl acetate (3×30 mL). The combined organic extracts are dried over magnesium sulfate, filtered and concentrated in vacuo to afford the desired product as a white solid. ISMS: MH⁺ 258.0

### Example 17

### Preparation of 1,7-diphenyl-4-aminoheptane hydrochloride (17)

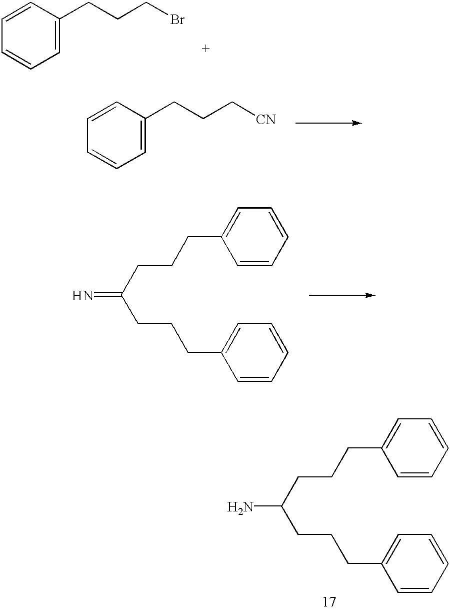

Magnesium (40.2 g, 1.65 mol) and anhydrous ether (3.2 L) are combined in a reaction vessel with stirring. A solution of 1-bromo-3-phenyl propane in 1.6 L of anhydrous ether is added to an addition funnel. The bromide solution is added dropwise to the stirring reaction vessel over a 1 hour period. Upon completion of addition, the mixture stirs for 1-2 hours. A solution of 4-phenylbutyronitrile (160 g, 1.1 mol) in anhydrous ether (2.4 L) is placed in the addition funnel. The solution is added to the reaction vessel over a 1 hour time period. Upon complete addition the solution is heated to reflux for 10 hours, and then stirs at room temperature for six hours. The reaction mixture is diluted with methanol (3.2 L) using an addition funnel. Sodium borohydride (83.4 g, 2.2 mol) is added in portions. Upon complete addition the reaction is stirred at room temperature for six hours. The reaction mixture is quenched by a slow addition of water (3.2 L). The mixture is diluted with ether (3.2 L) and water (1.6 L). The ether layer is separated and the aqueous layer is extracted twice with ether (3.2 L×2). The combined ether extracts are washed once with sodium chloride solution, dried, filtered, and concentrated in vacuo to give the crude product. This product is diluted in ether (1.2 L) and acidified by slow addition of 1M HCl (1.2 L). The mixture stirs for one hour and is concentrated in vacuo. The resulting precipitate is diluted with acetonitrile and is stirred for 16 hours. The desired 1,7-diphenyl-4-aminoheptane hydrochloride is collected by filtration.

### Example 18

### Preparation of N-(tert-butoxycarbonyl)-trans-4-(aminomethyl)- 1-cyclohexanecarboxylic acid [4-phenyl-1-(3-phenyl-propyl)-butyl]-amide (18):

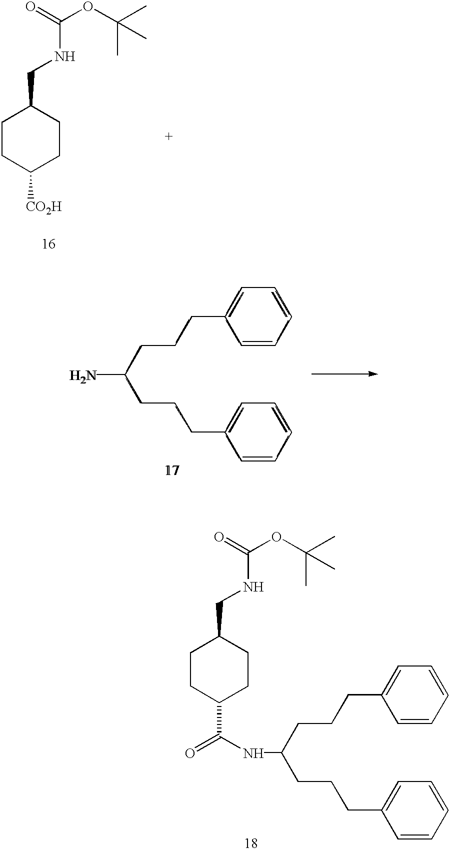

N-(tert-Butoxycarbonyl)-trans-4-(aminomethyl)-1-cyclohexanecarboxylic acid (16) (2.00 g; 7.77 mmol) is dissolved in methylene chloride (60 mL) at ambient temperature. 1,7-Diphenyl-4-aminoheptane hydrochloride (17) (2.83 g; 9.33 mmol), N,N-diisopropylethylamine (3.21 g; 24.9 mmol) and PyBOP(4.85 g; 9.33 mmol) are added sequentially. The reaction is stirred 27.5 hours at room temperature, then concentrated under reduced pressure. The residue is purified via silica gel chromatography (20%→40% ethyl acetate in hexanes) affording the desired product as a solid. ISMS: MH⁺ 507.4

### Example 19

### Preparation of trans-4-(aminomethyl)-1-cyclohexanecarboxylic acid [4-phenyl-1-(3-phenyl-propyl)-butyl]-amide (19):

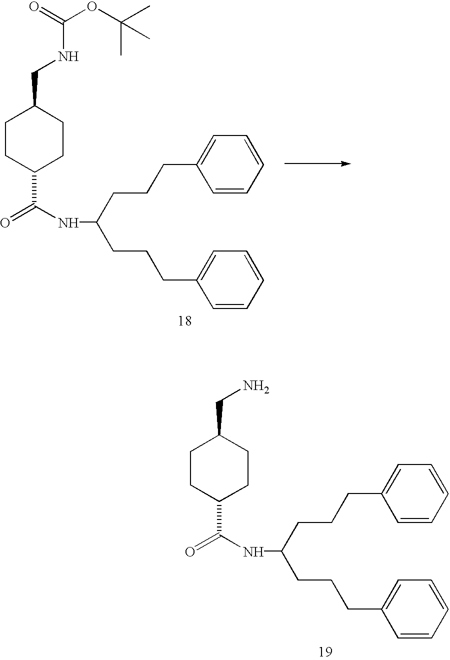

N-(tert-Butoxycarbonyl)-trans-4-(aminomethyl)-1-cyclohexanecarboxylic acid [4-phenyl-1-(3-phenyl-propyl)-butyl]-amide (18) (1.76 g; 3.47 mmol) is dissolved in methylene chloride (30 mL) at ambient temperature. Trifluoroacetic acid (15 mL) is added in a slow stream, and the solution is stirred for 6 hours at ambient temperature. The solution is concentrated in vacuo at 40° C. The residue is dissolved in methylene chloride (200 mL) and poured onto saturated sodium bicarbonate solution. The pH is adjusted to 9 with saturated potassium carbonate solution. The mixture is shaken and the layers separated. The water layer is extracted with methylene chloride (3×50 mL). The combined organic extracts are washed with water, dried over MgSO₄, filtered, and concentrated in vacuo affording the desired product as a solid. ISMS: MH⁺ 407.4

### Example 20

### Preparation of 1-{(R)-1-[2-hydroxy-3-(quinolin-5-yloxy)-propyl]}-4-(carboxylmethyl)piperazine ethyl ester (20):

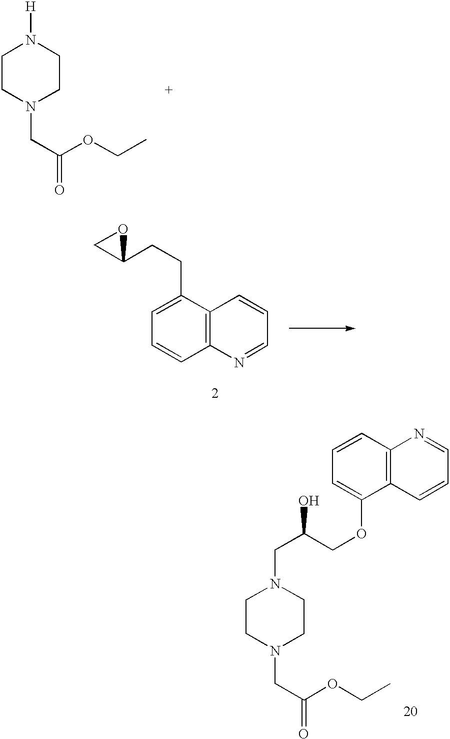

1-(Carboxylmethyl)piperazine ethyl ester (1.71 g; 9.94 mmol) is dissolved in ethanol (200 mL) at ambient temperature. (R)-5-Oxiranylmethoxy-quinoline (2) (2.00 g; 9.94 mmol) is added, then the mixture is refluxed for 18 hours. After cooling to ambient temperature, the solution is concentrated in vacuo at 40° C. The residue is purified via silica gel chromatography with gradient elution (70%→100% acetone in hexanes, then 5% ethanol in acetone) affording the desired product as an oil. ESMS: MH⁺ 374.2

### Example 21

### Preparation of 1-{(R)-1-[2-hydroxy-3-(quinolin-5-yloxy)-propyl]}-4-(carboxylmethyl)piperazine lithium salt (21):

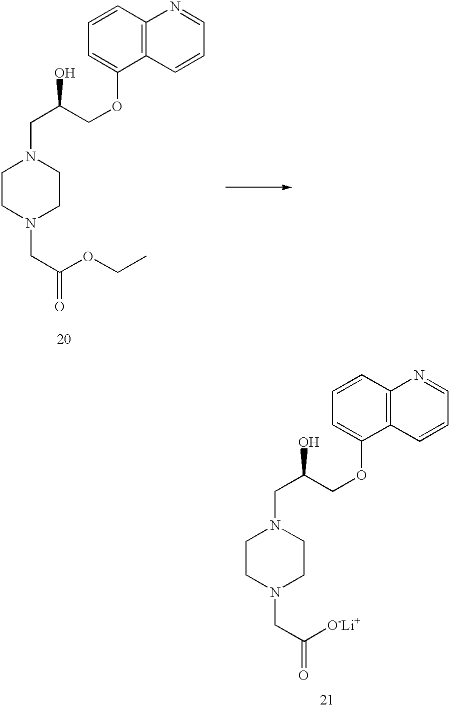

1-{(R)-1-[2-Hydroxy-3-(quinolin-5-yloxy)-propyl]}-5-(carboxylmethyl)piperazine ethyl ester (20) (1.34 g; 3.59 mmol) is dissolved in 40 mL of 2:2:1 tetrahydrofuran: water: methanol. Lithium hydroxide (90.2 mg; 3.77 mmol) is added and the solution stirred for 17 hours at ambient temperature. The solution is concentrated at reduced pressure to afford the desired product as a solid.

### Example 22

### Preparation of N-{1-[2-(R)-hydroxy-3-(quinolin-5-yloxy)-propyl]-piperazine-4-acetyl}-trans-4-aminomethyl-1-cyclohexanecarboxylic acid [4-phenyl-1-(3-phenyl-propyl)-butyl]-amide (22):

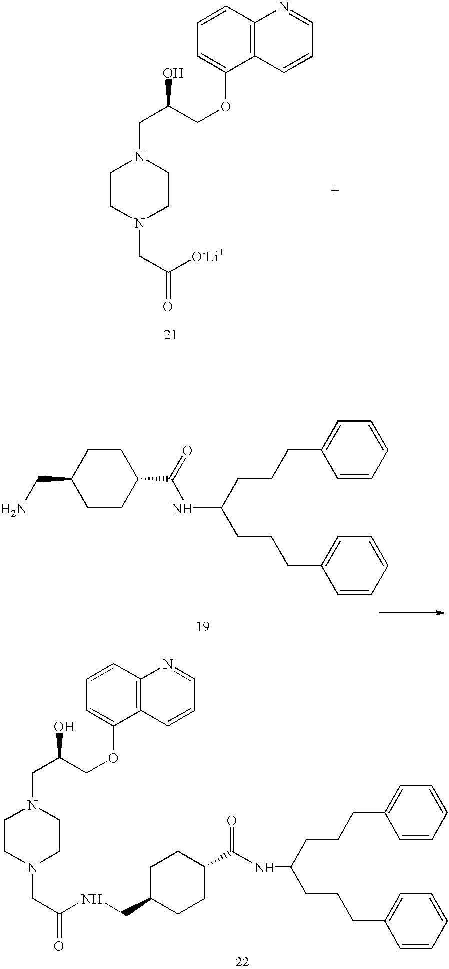

1-{(R)-1-[2-Hydroxy-3-(quinolin-5-yloxy)-propyl]}-4-(carboxylmethyl)piperazine lithium salt (21) (100 mg; 0.285 mmol) is dissolved in N,N-dimethylformamide (3 mL) at ambient temperature. trans-4-Aminomethyl-1-cyclohexanecarboxylic acid [4-phenyl-1-(3-phenyl-propyl)-butyl]-amide (19) (138.9 mg; 0.342 mmol), N,N-diisopropylethylamine (80.9 mg; 0.626 mmol) and PyBOP (177.7 mg; 0.342 mmol) are added sequentially. The reaction is stirred for 46 hours at ambient temperature. Water (3 mL) is added and the mixture shaken. The layers are separated and the water layer extracted with methylene chloride (2×2 mL). The combined methylene chloride extracts are dried over magnesium sulfate, filtered and concentrated under reduced pressure. The residue is purified via silica gel chromatography (50%→100% acetone in hexanes, then 5%→20% ethanol in acetone) affording the desired product. ESMS: MH⁺ 734.6

### Example 23

### Preparation of 1-(tert-butoxycarbonyl)-piperidine-4-carboxylic acid [4-phenyl-1-(3-phenyl-propyl)-butyl]-amide (23):

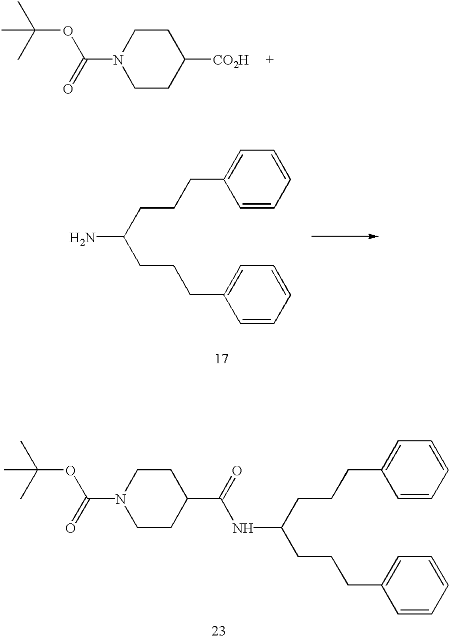

1-tert-Butoxycarbonyl-piperidine-4-carboxylic acid (4.00 g; 17.4 mmol) is dissolved in methylene chloride (125 mL) at ambient temperature. 1,7-Diphenyl-4-aminoheptane hydrochloride (17) (6.36 g; 20.9 mmol), diisopropylethylamine (7.27 g; 55.8 mmol), and PyBOP (10.89 g; 20.9 mmol) are added sequentially. The mixture is stirred for 14 hours at ambient temperature then concentrated in vacuo at 40° C. The residue is purified via silica gel chromatography with gradient elution (20%→40% ethyl acetate in hexanes) affording the desired product as an oil.

### Example 24

### Preparation of piperidine-4-carboxylic acid [4-phenyl-1-(3-phenyl-propyl)-butyl]-amide (24):

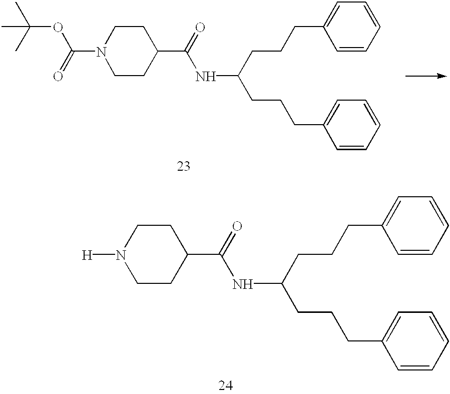

1-(tert-Butoxycarbonyl)-piperidine-4-carboxylic acid [4-phenyl-1-(3-phenyl-propyl)-butyl]-amide (23) (8.17 g; 17.1 mmol) is dissolved in methylene chloride (60 mL) at ambient temperature. Trifluoroacetic acid (40 mL) is added in a slow stream, and the solution is stirred for 2 hours at ambient temperature. The solution is concentrated in vacuo at 40° C. The residue is dissolved in methylene chloride (400 mL) and poured onto saturated sodium bicarbonate solution. The pH is adjusted to 9 with saturated potassium carbonate solution. The mixture is shaken and the layers separated. The water layer is extracted with methylene chloride (3×100 mL). The combined organic extracts are washed with water, dried over MgSO₄, filtered, and concentrated in vacuo affording the desired product (5.91 g) as a white solid. ESMS: MH⁺ 379.0

### Example 25

### Preparation of 1-[N-tert-butoxycarbonyl-3-(3-pyridyl)-alanyl]-piperidine 4-carboxylic acid [4-phenyl-1-(3-phenyl-propyl)-butyl]-amide (25):

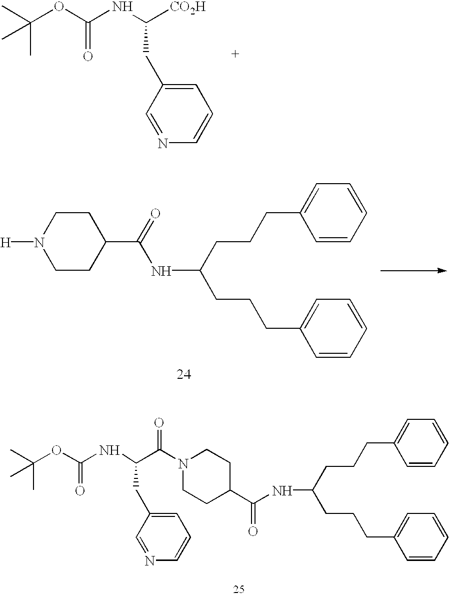

N-tert-Butoxycarbonyl-3-(3-pyridyl)-alanine (1.00 g; 3.76 mmol) is dissolved in methylene chloride (25 mL) at ambient temperature. Piperidine-4-carboxylic acid [4-phenyl-1-(3-phenyl-propyl)-butyl]-amide (24) (1.71 g; 4.51 mmol), N,N-diisopropylethylamine (1.07 g; 8.26 mmol) and PyBOP (2.34 g; 4.51 mmol) are added sequentially. The reaction is stirred for 6.5 hours at room temperature, then concentrated under reduced pressure. The residue is purified via silica gel chromatography (90% ethyl acetate in hexanes, then 50% acetone in hexanes) affording the desired product as a solid. ESMS: MH⁺627.4

### Example 26

### Preparation of 1-[3-(pyridyl)-alanyl]-piperidine-4-carboxylic acid [4-phenyl-1-(3-phenyl-propyl)-butyl]-amide (26):

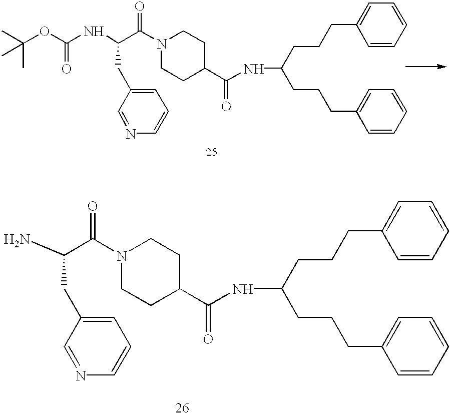

1-[N-tert-Butoxycarbonyl-3-(3-pyridyl)-alanyl]-piperidine-4-carboxylic acid [4-phenyl-1-(3-phenyl-propyl)-butyl]-amide (25) (2.34 g; 3.73 mmol) is dissolved in methylene chloride (40 mL) at ambient temperature. Trifluoroacetic acid (20 mL) is added in a slow stream, and the solution is stirred for 1.5 hours at ambient temperature. The solution is concentrated in vacuo at 40° C. The residue is dissolved in methylene chloride (300 mL) and poured onto saturated sodium bicarbonate solution. The pH is adjusted to 9 with saturated potassium carbonate solution. The mixture is shaken and the layers separated. The water layer is extracted with methylene chloride (3×75 mL). The combined organic extracts are washed with water, dried over MgSO₄, filtered, and concentrated in vacuo affording the desired product (1.59 g) as an oil. ESMS: MH⁺527.2

### Example 27

### Preparation of 1-(N-{1-[2-(R)-hydroxy-3-(quinolin-5-yloxy)-propyl]-piperazine-4-acetyl}-[3-(3-pyridyl)-alanyl])-piperidine-4-carboxylic acid [4-phenyl-1(3-phenyl-propyl)-butyl]-amide (27):

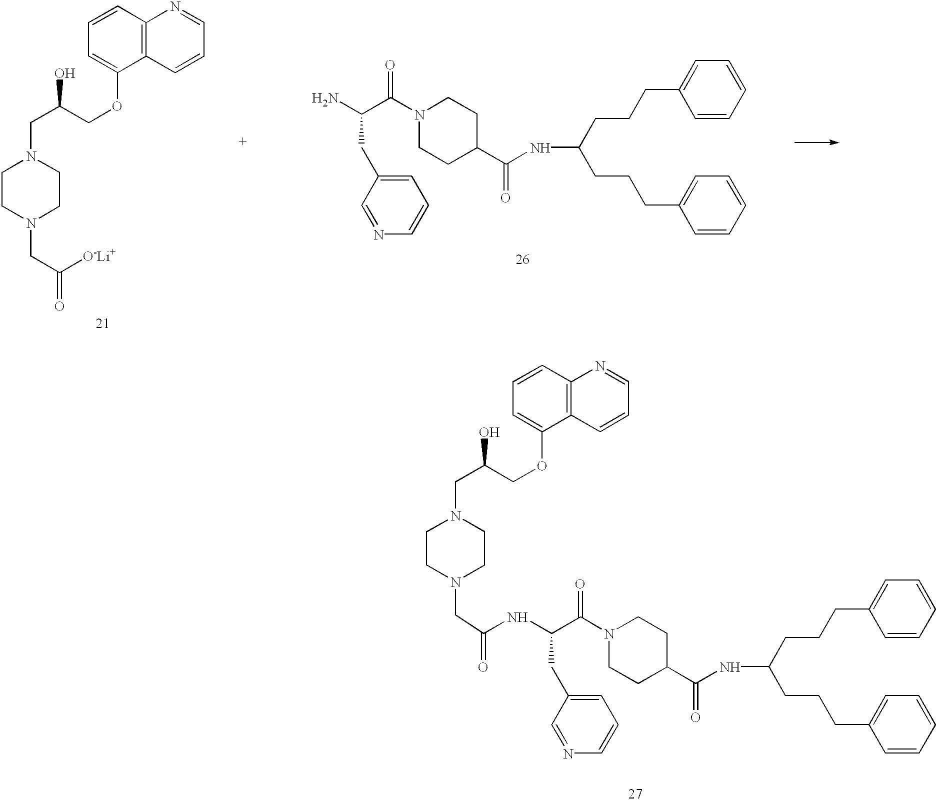

1-{(R)-1-[2-Hydroxy-3-(quinolin-5-yloxy)-propyl]}-4-(carboxylmethyl)piperazine lithium salt (21) (100 mg; 0.285 mmol) is dissolved in N,N-dimethylformamide (3 mL) at ambient temperature. 1-[3-(3-Pyridyl)-alanyl]-piperidine-4-carboxylic acid [4-phenyl-1-(3-phenyl-propyl)-butyl]-amide (26) (179.9 mg; 0.342 mmol), N,N-diisopropylethylamine (80.9 mg; 0.626 mmol) and PyBOP (177.7 mg; 0.342 mmol) are added sequentially. The reaction is stirred for 46 hours at ambient temperature. Water (3 mL) is added and the mixture shaken. The layers are separated and the water layer extracted with methylene chloride (2×2mL). The combined methylene chloride extracts are dried over magnesium sulfate, filtered and concentrated under reduced pressure. The residue is purified via silica gel chromatography (50%→100% acetone in hexanes, then 5%→20% ethanol in acetone) affording the desired product. ESMS: MH⁺854.6

### Example 28

### Activity of the Compounds

Accumulation Index of various compounds prepared above was tested according to the method in Reference Example 3. The results are in Table 3.

TABLE 3
Accumulation Index of the Active Compounds

| Compound | Accumulation Index |
|---|---|
| Example7 | 9 |
| Example 10 | 8 |
| Example 15 | 6 |
| Example 22 | 7 |
| Example 27 | 8 |

### Example 29

### Oral Composition for the Active Compound of this Invention

A composition for oral administration is prepared by reducing an active compound according to this invention to a No. 60 powder. Starch and magnesium stearate are passed through a No. 60 bolting cloth onto the powder. The combined ingredients are mixed for 10 minutes and filled into a hard shell capsule of a suitable size at a fill weight of 100 mg per capsule. The capsule contains the following composition:

| | |
|---|---|
| Active Compound | 5 mg |
| Starch | 88 mg |
| Magnesium Stearate | 7 mg |

### Example 30

### Oral Composition for the Active Compound of this Invention with a Chemotherapeutic Agent

A mixture of vinblastine and an active compound of this invention is reduced to a No. 60 powder. Lactose and magnesium stearate are passed through a No. 60 bolting cloth onto the powder. The combined ingredients are mixed for 10 minutes. and then filled into a No. 1 dry gelatin capsule. Each capsule contains the following composition:

| | |
|---|---|
| Active Compound | 5 mg |
| Vinblastine | 5 mg |
| Lactose | 580 mg |
| Magnesium Stearate | 10 mg |

### Example 31

### Parenteral Composition for the Active Compound of this Invention

An active compound according to this invention (1 mg) is dissolved in 1 mL of a solution of 10% cremaphor, 10% ethanol, and 80% water. The solution is sterilized by filtration.

### Example 32

### Parenteral Composition for the Active Compound of this Invention

A sufficient amount of an active compound according to this invention and TAXOL® are dissolved in a 0.9% sodium chloride solution such that the resulting mixture contains 0.9 mg/mL of the active compound of this invention and 1.2 mg/mL TAXOL®.

A sufficient amount of the solution to deliver 135 mg/sq m TAXOL® is administered intravenously over 24 hours to a patient suffering from ovarian cancer.

What is claimed is:

1. An active compound selected from the group consisting of a structure: 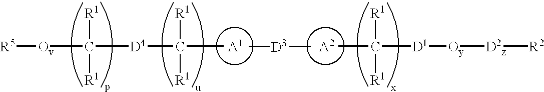
wherein A¹ and A² are each independently selected from the group consisting of carbocyclic groups, substituted carbocyclic groups, heterocyclic groups, substituted heterocyclic groups, aromatic groups, substituted aromatic groups, heteroaromatic groups, and substituted heteroaromatic groups;
each R¹ is independently selected from the group consisting of a hydrogen atom, a hydroxyl group, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
x is 0 to about 10;
R² is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
D¹ and D² are each independently selected from the group consisting of —C(O)— and —NR³—,
wherein R³ is selected from the group consisting of a hydrogen atom and R², and with the proviso that optionally, R² and R³ may be bonded together thereby forming a ring selected from the group consisting of heterocyclic groups and substituted heterocyclic groups;
y is 0 or 1 and z is 0 or 1;
D³ is selected from the group consisting of a bond, hydrocarbon groups, substituted hydrocarbon groups, heterogeneous groups, and substituted heterogeneous groups; with the proviso that substituted hydrocarbon groups for D³ are not hydrocarbon groups having only one —C(O)— group,
u is 0 to about 10, p is 0 to about 10, and v is 0 or 1;
D⁴ is selected from the group consisting of —S(O)₂—, —C(O)—, and —CR¹(OH)—; and
R⁵ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group; and
an optical isomer, a diastereomer, an enantiomer, a pharmaceutically-acceptable salt, a biohydrolyzable amide, a biohydrolyzable ester, and a biohydrolyzable imide of the structure.

2. The compound of claim 1, wherein A¹ and A² are each independently substituted heterocyclic groups.

3. The compound of claim 2, wherein D³ is selected from the group consisting of a a bond, —(CR¹₂)_{w}—NR⁴—(CR¹₂)_{w}—, —(CR¹₂)₂—C(O)—(CR¹₂)_{w}, —NR⁴—(CR¹₂)_{w}—, and —(CR¹₂)₂—C(O)—(CR¹₂)_{w}—NR⁴—(CR¹₂)_{w}—C(O)—(CR¹₂)_{w}—; wherein each w is independently 0 to about 10, and R⁴ is selected from the group consisting of a hydrogen atom, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group.

4. The compound of claim 3, wherein D¹ is —C(O)—, x is 0, and y is 0.

5. The compound of claim 4, wherein R² is selected from the group consisting of: 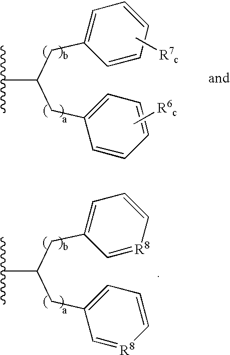
wherein [symbol] denotes a point of attachment, a is about 3 to about 10 and b is about 3 to about 10;
R⁶ and R⁷ are each independently selected from the group consisting of hydrocarbon groups and substituted hydrocarbon groups; and
each R⁸ is independently selected from the group consisting of CH and a heteroatom;

6. The compound of claim 5, wherein D⁴ is —CR¹(OH)— and v is 1.

7. The compound of claim 6, wherein R⁵ has the formula 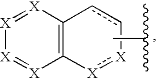
wherein [symbol] denotes a point of attachment and each X is independently selected from the group consisting of CH and a heteroatom, with the proviso that at least one X is a heteroatom.

8. The compound of claim 7, wherein the compound has a structure selected from the group consisting of:

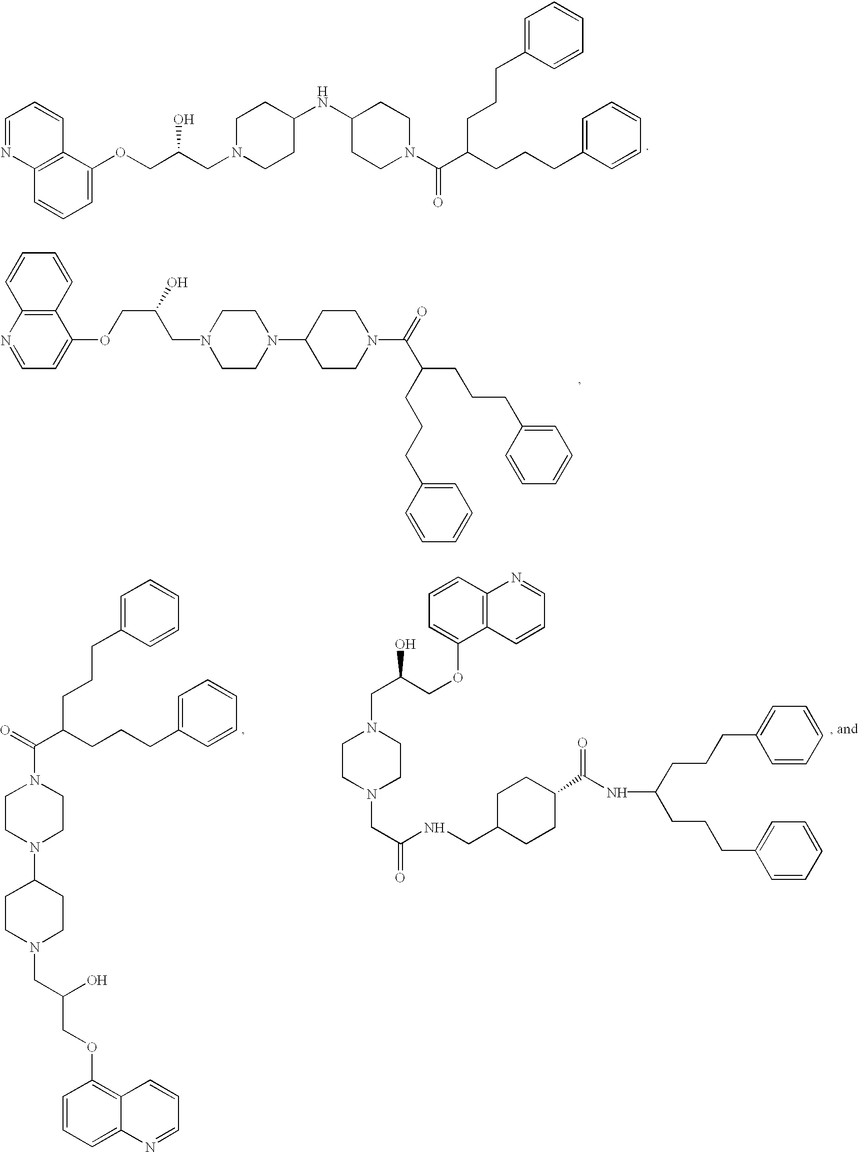

9. A composition for treating multidrug resistance comprising:
(A) a compound selected from the group consisting of a structure 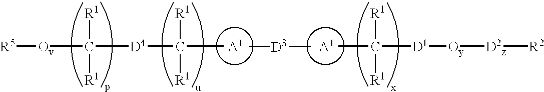
wherein A¹ and A² are each independently selected from the group consisting of carbocyclic groups, substituted carbocyclic groups, heterocyclic groups, substituted heterocyclic groups, aromatic groups, substituted aromatic groups, heteroaromatic groups, and substituted heteroaromatic groups;
each R¹ is independently selected from the group consisting of a hydrogen atom, a hydroxyl group, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
x is 0 to about 10;
R² is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
D¹ and D² are each independently selected from the group consisting of —C(O)— and —NR³—,
wherein R³ is selected from the group consisting of a hydrogen atom and R², and with the proviso that optionally, R² and R³ may be bonded together thereby forming a ring selected from the group consisting of heterocyclic groups and substituted heterocyclic groups;
y is 0 or 1 and z is 0 or 1;
D³ is selected from the group consisting of a bond, hydrocarbon groups, substituted hydrocarbon groups, heterogeneous groups, and substituted heterogeneous groups; with the proviso that substituted hydrocarbon groups for D³ are not hydrocarbon groups having only one —C(O)— group,
u is 0 to about 10, p is 0 to about 10, and v is 0 or 1;
D⁴ is selected from the group consisting of —S(O)₂—, —C(O)—, and —CR¹(OH)—; and
R⁵ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group; and
an optical isomer, a diastereomer, an enantiomer, a pharmaceutically-acceptable salt, a biohydrolyzable amide, a biohydrolyzable ester, and a biohydrolyzable imide of the structure; and
(B) a carrier.

10. The composition of claim 9, further comprising: component (C) a therapeutic agent selected from the group consisting of (i) a cancer therapeutic agent, (ii) an antibacterial agent, (iii) an antiviral agent, (iv) an antifungal agent, and combinations thereof.

11. A method for inhibiting transport protein activity comprising administering, to a subject, a compound selected from the group consisting of a structure: 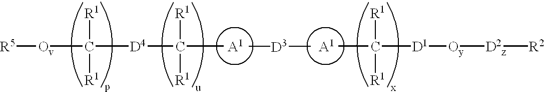
wherein A¹ and A² are each independently selected from the group consisting of carbocyclic groups, substituted carbocyclic groups, heterocyclic groups, substituted heterocyclic groups, aromatic groups, substituted aromatic groups, heteroaromatic groups, and substituted heteroaromatic groups;
each R¹ is independently selected from the group consisting of a hydrogen atom, a hydroxyl group, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
x is 0 to about 10;
R² is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
D¹ and D² are each independently selected from the group consisting of—C(O)— and —NR³—,
wherein R³ is selected from the group consisting of a hydrogen atom and R², and with the proviso that optionally, R² and R³ may be bonded together thereby forming a ring selected from the group consisting of heterocyclic groups and substituted heterocyclic groups;
y is 0 or 1 and z is 0 or 1;
D³ is selected from the group consisting of a bond, hydrocarbon groups, substituted hydrocarbon groups, heterogeneous groups, and substituted heterogeneous groups; with the proviso that substituted hydrocarbon groups for D³ are not hydrocarbon groups having only one —C(O)— group,
u is 0 to about 10, p is 0 to about 10, and v is 0 or 1;
D⁴ is selected from the group consisting of —S(O)₂—, —C(O)—, and —CR¹(OH)—; and
R⁵ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group; and
an optical isomer, a diastereomer, an enantiomer, a pharmaceutically-acceptable salt, a biohydrolyzable amide, a biohydrolyzable ester, and a biohydrolyzable imide of the structure; and combinations thereof.

12. The method of claim 11, further comprising coadministering component (C) a therapeutic agent.

13. The method of claim 12, wherein component (C) is selected from the group consisting of (i) a cancer therapeutic agent, (ii) an antibacterial agent, (iii) an antiviral agent, (iv) an antifungal agent, and combinations thereof.

14. The method of claim 12, wherein component (C) is coadministered at a time selected from the group consisting of before, during, and after administration of component (A); and combinations thereof.
